(19) United States
(12) Patent Application Publication
(10) Pub. No.: US 2020/0227141 A1
(43) Pub. Date: Jul. 16, 2020

(54) Method and Systems for detection of biomarkers in response to intoxicant consumption

(71) Applicants: Itzhak Kurek, San Francisco, CA (US); Robert McKee, Austin, TX (US)

(72) Inventors: Itzhak Kurek, San Francisco, CA (US); Robert McKee, Austin, TX (US)

(21) Appl. No.: 15/973,459

(22) Filed: May 7, 2018

Related U.S. Application Data
(60) Provisional application No. 62/502,023, filed on May 5, 2017.

(51) Int. Cl. G16B 45/00; G16B 5/00; G16B 20/00; G16C 20/20; G16C 20/80

(52) U.S. Cl. CPC G16B 45/00; G16B 5/00; G16C 20/80; G16C 20/20; G16B 20/00

(57) ABSTRACT

Disclosed are methods and systems for detecting biomarkers indicative of a time of consumption of a substance and indicative of the effect of a substance on a person.

### CROSS-REFERENCE TO RELATED APPLICATIONS

This application claims priority to and the benefit of the filing of U.S. Provisional Patent Application Ser. No. 62/502,023 entitled “Method and Systems for detection of biomarkers in response to intoxicant consumption”, by Itzhak Kurek and Robert McKee, filed on May 5, 2017, and the specification, figures, and claims thereof are incorporated herein by reference.

### BACKGROUND OF THE INVENTION

### Field of the Invention

Embodiments of the present invention relate to the detection of biomarkers.

### Description of Related Art

The increasing usage of cannabis from different cultivated varieties, the lack of standards for plant contents and dosage, and the high number of active components significantly complicate the conventional approach of identification of a single active ingredient. Furthermore, conventional approaches make it difficult to identify how a person is reacting to cannabis.

There is a need for more accurate systems and methods to identify targeted and untargeted biomarkers.

### SUMMARY

This summary is provided to introduce a selection of concepts in a simplified form that are further described below in the Detailed Description. This summary is not intended to exclusively identify key features or essential features of the claimed subject matter, nor is it intended as an aid in determining the scope of the claimed subject matter.

Embodiments are directed to methods, devices, and systems for detecting untargeted and targeted biomarkers. An example method may include receiving, by the one or more computing devices, a genetic and chemical profile of a cultivar; receiving, by the one or more computing devices, a user profile, wherein the user profile may comprise method of consumption and demographic data of a consumer; receiving, by the one or more computing devices, a metabolomic profile of said consumer, wherein the metabolomic profile comprises metabolomics data acquired at a time prior to consumption of said cultivar, at a time of consumption of said cultivar, and at a time after the consumption of said cultivar; and comparing, by the one or more computing devices, said metabolomics data acquired at a time prior to consumption of said cultivar to said metabolomics data acquired at a time of consumption of said cultivar, and said metabolomics data acquired at a time after the consumption of said cultivar. Example methods for detecting biomarkers may further include generating, by the one or more computing devices, from said comparing step, a differences profile comprising differences recognized by the one or more computing devices in said metabolomics data acquired at said time prior to consumption of said cultivar to said metabolomics data acquired at said time of consumption of said cultivar, and said metabolomics data acquired at said time after the consumption of said cultivar; and calculating, by the one or more computing devices, a correlation between said time of consumption of said cultivar and one or more data points from said genetic and chemical profile of said cultivar, said user profile, or said metabolomics data of said differences profile. The metabolomic profile may include a plurality of ratios of biomarkers within the metabolomic profile.

According to other examples, the calculating step may further comprise training a machine learning algorithm to estimate said time of consumption of said cultivar using said genetic and chemical profile of said cultivar, said user profile, and said metabolomics data of said differences profile; using said machine learning algorithm to estimate an unknown time of consumption of an unknown cultivar using new data comprising a plurality of data points from said user profile, and said metabolomics data of said differences profile; and generating a confidence score confidence score that represents the likelihood that a consumer consumed marijuana or another intoxicant within a specific time frame. The method may further comprise a step of feature extraction.

These and other features and advantages will be apparent from a reading of the following detailed description and a review of the associated drawings. It is to be understood that both the foregoing general description and the following detailed description are explanatory and do not restrict aspects as claimed.

### BRIEF DESCRIPTION OF THE DRAWINGS

The drawings, which are incorporated herein, illustrate one or more embodiments of the present invention, thus helping to better explain one or more aspects of the one or more embodiments. As such, the drawings are not to be construed as limiting any particular aspect of any embodiment of the invention. In the drawings:

FIG. 1 illustrates a method for detecting biomarkers.

FIG. 2 depicts a system for creating a metadata profile, comprising inputs utilized by processes that then produce outputs.

FIG. 3 illustrates a schematic presentation of the bioinformatics platform processing overview of consumer sample pre-consumption of intoxicants.

FIG. 4 illustrates a processing overview of consumer samples post-intoxicant consumption.

FIG. 5 depicts a table representing metabolomics data produced from the processes.

FIG. 6 depicts a system for identifying time relevant or pharmaceutical relevant biomarkers.

FIG. 7 illustrates a block diagram of an example computing device, in accordance with at least one embodiment.

FIG. 8 illustrates a system environment diagram, in which various embodiments may be implemented.

FIG. 9 depicts a flow chart representing a metabolomics module.

FIG. 10 depicts a flow chart representing a consumer module.

FIG. 11 depicts a flow chart representing a cultivar module.

FIG. 12 illustrates a flow diagram of neuropathy patients trial study.

FIG. 13 illustration of time series data from trial study.

FIG. 14 illustrates a sampling schedule of an experiment of an embodiment.

FIG. 15 illustrates the effect of cannabis consumption on the levels of Octanoic acid and Ribulose 5-phosphate (Ru5P).

FIG. 16 illustrates the effects of cannabis on the levels of Octanoic acid and Ribulose 5-phosphate (Ru5P).

FIG. 17 illustrates the effect of cannabis consumption on the levels of Dyphylline and Guanine.

FIG. 18 illustrates the effect of cannabis consumption on the levels of N-acetyl aspartate and creatine.

FIG. 19 illustrates time dependent levels of Alanine, Inosine and Hydroxyproline.

### DETAILED DESCRIPTION

The term biomarker may include a metabolite that exhibits statistically significant values difference between iMPpr (pre-consumption) to one or more time points.

The term ratio may include a ratio of 2 or more metabolites that exhibits statistically significant differences between 2 or more time points.

Cultivar, substance, and intoxicant may be used interchangeably to represent a plant, a drug, marijuana, or chemical intoxicants such as cocaine, crystal meth, or opiates.

The term chemical profile (chemovar) may include chemical analysis for chemotaxonomic mapping of plant or microorganism varieties with differences in secondary metabolite content and distribution.

The term genetic profile may include unique DNA pattern based on a set of sequence variations that differentiates individuals of a species, such as cannabis cultivars.

The term Cultivar (cultivated variety) may include the basic classification of a plant that is uniform and stable in its characteristics, can be reproduced toward defined goals and is not subject to extinction. Cultivar and intoxicant may be used interchangeably to represent a plant, a drug, marijuana, or chemical intoxicants such as cocaine, crystal meth, or opiates.

The term user profile may include personal data associated with a specific individual who may be a consumer, candidate or participant in a research study.

The term method of consumption may include cannabis intake includes 3 routes: 1) inhalation; 2) ingestion and; 3) skin absorption. 1) Inhalation consumption forms are: a) smoking of combusted, dried flowers of the cannabis plant; b) vaporizing of cannabis flower or extract or concentrate in a precise temperature that allows therapeutic ingredients to phase-change into a gas or vapor and be extracted without burning the plant; and c) dabbing, the flash vaporization method of concentrates (shatter, wax, BHO, oil, etc.) which are more potent than cannabis flowers. 2) Ingestion: consumption of food-based edibles in solid or liquid form that have been formulated with cannabis-infused butter, infused oil or other cannabis-infused edibles such as candies, cookies and brownies. 3) Skin absorption: the route by which cannabis-infused topicals in the forms of lotions, balms, oils, lubricants and transdermal patches.

The term demographic data may include socioeconomic characteristics of the tested users expressed statistically and includes categories such as: sex, age, race, income, education and employment. The population can be specific to geographic location and associated with time.

The term metabolite profile/metabolic profile may include profile of pre-defined metabolites belonging to a class of compounds such as polar lipids, isoprenoids, carbohydrates, or members of particular pathways.

The term metabolome may include a complete set of metabolites that consists of low-molecular-weigh intermediates and products of the metabolism process in a biological system.

The term metabolomics/metabolomic profiling may include a global profiling process that measures multiple metabolite concentrations and fluctuations reflecting the dynamic response of a biological cell, tissue, organ or organism in response to

drugs, diet, lifestyle, environment, stimuli and genetic modulations.

The term metabolomics Data Acquisition may include the process of comprehensive identification and quantification of a metabolite set from a sample of a biological system using the analytical platforms nuclear magnetic resonance (NMR) spectroscopy and or mass spectrometry (MS). To reduce sample complexity and to minimize ionization suppression effects, MS requires a previous separation step, using a hyphenated separation technique, such as gas chromatography (GC), high-performance liquid chromatography (HPLC) or ultra-performance liquid chromatography (UPLC), and capillary electrophoresis (CE).

The term metabolomics Data Processing may include the step in which the acquired raw data are submitted to an analytical platform for conversion into a numerical format that can be used for downstream statistical analysis. NMR data processing includes phasing, baseline correction, alignment, and normalization by software and algorithms, such as PERCH (PERCH Solution Ltd.), Chenomx NMR Suite (Chenomx Inc.), MestReNova (MestreLab Research), MetaboLab , AutoFit, TopSpin (Bruker Corp.), and MATLAB (The MathWorks Inc.). Hyphenated MS techniques data processing includes spectral deconvolution, dataset creation, grouping, alignment, filling data gaps, normalization, and data transformation using softwares such as XCMS, Mass Profiler Professional (MPP, Agilent Technologies), MZmine, MetAlign, MassLynx (Waters Corp.).

The term metabolomics Statistical Analysis may include the process that reveals discriminant metabolites between control and test samples using chemometric tools for sample overview and classification include: 1) multivariate analyses unsupervised methods, such as principal component analysis (PCA), and 2) supervised methods, such as partial least square discriminant analysis (PLS-DA) and orthogonal projections to latent structure discriminant analysis (OPLS-DA). Univariate analysis based on Student's t-test, Mann-Whitney U test, etc. can be used to confirm multivariate results.

The term metabolite identification may include the process that identifies putative metabolites and reveals the identity based on matching features from sample spectra against a reference spectral database and libraries, such as HMDB, KEGG, PubChem, Metlin, MassBank, LIPID MAPS, ChEBI, MMD, BioMagResBank, MetaboID, and Chenomx NMR Suite (Chenomx Inc.). Highly efficiency metabolite identification can be achieved by: (1) context-specific spectral database for biologically and biochemically possible candidates, and (2) incorporation of prior knowledge based on spectral dependencies, biochemical connectivities and biological relationships.

The term machine learning, or alternatively described as machine learning database, or deep learning (as described below) may include machine learning methods may include supervised learning, un-supervised learning, reinforcement learning, decision tree learning, association rule learning, artificial neural networks, deep learning, inductive logic programming, support vector machines, clustering, Bayesian networks, reinforcement learning, similarity and metric learning, genetic algorithms, rule-based machine learning, learning classifiers, recurrent neural networks, and adversarial neural networks.

The term deep learning may include neural networks and deep learning architectures such as deep neural networks, convolutional deep neural networks, deep belief networks and recurrent neural networks. Deep learning algorithms may use a cascade of many layers of nonlinear processing units for feature extraction and transformation. Each successive layer may use the output from the previous layer as input. The algorithms may be supervised or unsupervised. Deep learning algorithms may be based on the unsupervised learning of multiple levels of features or representations of the data.

Higher level features may be derived from lower level features to form a hierarchical representation. Deep learning algorithms may learn multiple levels of representations that correspond to different levels of abstraction; the levels form a hierarchy of concepts. Deep learning algorithms may comprise an output layer and one or more hidden layers, and training the deep learning algorithms may include: training the output layer by minimizing a loss function given the optimal set of assignments; and training the hidden layers through backpropagation.

The term feature extraction may include feature extraction starts from an initial set of measured data and builds derived values (features) intended to be informative and non-redundant, facilitating the subsequent learning and generalization steps, and in some cases leading to better human interpretations.

The term Time of consumption may include unknown time and known time of consumption of an intoxicant or other consumable matter.

The term Confidence score may generally describe a system that may train a machine learning algorithm that is configured to receive genetic and chemical profile of a cultivar, method of consumption and demographic data of a consumer, metabolomics data acquired at a time prior to consumption of said cultivar, at a time of consumption of said cultivar, and at a time after the consumption of said cultivar and estimate unknown time of consumption of an unknown cultivar and, for each estimate, generate a confidence score that represents the likelihood that a consumer consumed marijuana or another intoxicant within a specific time frame.

The term impact value may include a value indicating if a person is cognitively impaired by a substance such as marijuana, opiates, narcotics, or alcohol. The impact value may also indicate relief or presence of symptoms relating to pain, anxiety, depression, insomnia, epilepsy, seizures, and combinations thereof.

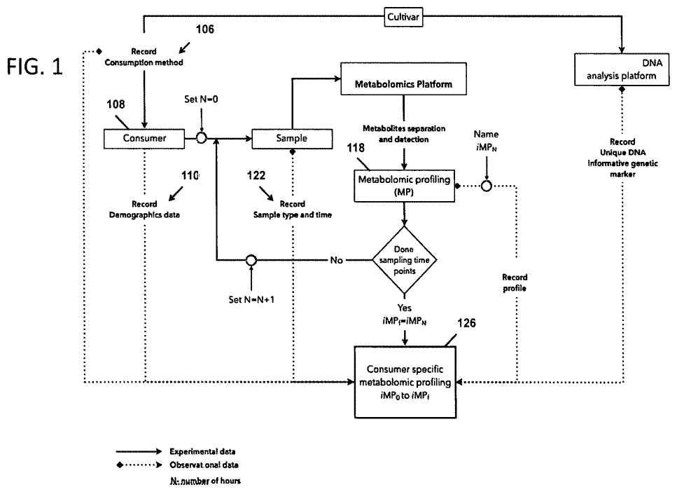

FIG. 1 illustrates a method for detecting biomarkers, or combinations of biomarkers present at certain ratios, that may indicate a time since consumption of a cultivar or other intoxicant. In some embodiments, the biomarkers, or combinations of biomarkers present at certain ratios, may indicate a desired affect resulting from consumption of a cultivar or other intoxicant. For example, a desired affect may include anxiety reduction, seizure reduction, pain reduction, migraine reduction, depression reduction, sedation, or intoxication.

As illustrated in FIG. 1, a genetic and/or chemical profile of a cultivar or toxicant may be created and stored in a machine readable format. The genetic and/or chemical profile in a machine readable format may then be utilized by a bioinformatics platform. A user profile 126 may be created and stored in a machine readable format. The user profile 126 in a machine readable format may then be utilized by a bioinformatics platform. The user profile may comprise data related to the method of consumption 106 of a cultivar or other intoxicant, the time of consumption 122, and demographic data 110 of the consumer 108 of the cultivar or intoxicant.

A metabolomic profile 118 may be created and stored in a machine readable format. The metabolomic profile in a machine readable format may then be utilized by a bioinformatics platform. The metabolomic profile may comprise metabolomics data from a user acquired at a time prior to consumption of a cultivar or intoxicant, at a time of consumption of the cultivar or intoxicant, and at one or more times after the consumption of the cultivar or intoxicant. The metabolomic profile may include ratios representing differences between biomarkers present in the metabolomics data.

A bioinformatics platform 126 may compare the metabolomics data to generate a differences profile comprising differences between the metabolomics data recognized by the one or more computing devices and machine learning algorithms. The differences profile may be created and stored in a machine readable format.

A differences profile may then be used by the bioinformatics platform to calculate a correlation between the time of consumption of the cultivar or intoxicant, and one or more data points from the genetic and chemical profile of the cultivar or intoxicant, the user profile, or the metabolomics data of the differences profile. The correlation may be created and stored in a machine readable format.

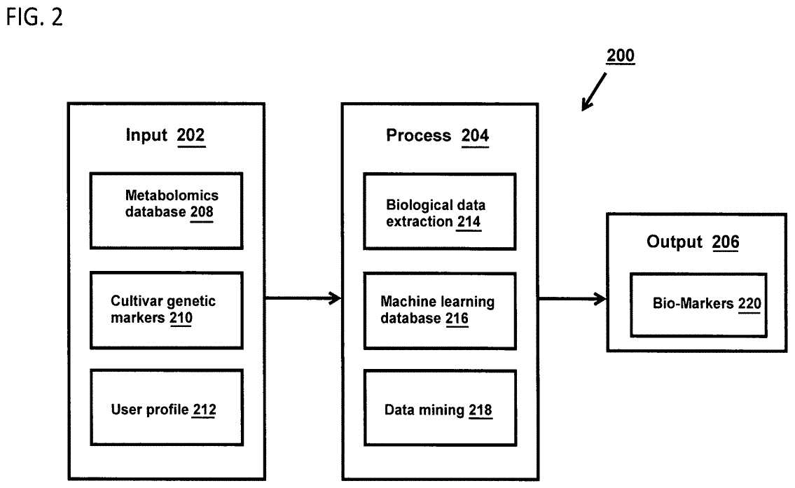

FIG. 2 depicts a system 200 for creating a metabolomics profile, comprising inputs 202 utilized by processes that then produce outputs 206. The inputs 202, may comprise metabolomics databases 208, cultivar genetic markers 210, and a user profile 212. The inputs 202 are utilized by the processes 204 to produce outputs 206. The metabolomics database 208 may comprise metabolomics data collected from saliva, hair, sweat, and or blood. However, other alternatives are contemplated. The cultivar genetic markers 210 may comprise data collected from Cannabis sativa, Cannabis indica, and hybrid Cannabis cultivars. However, other alternatives are contemplated. The user profile 212 may comprise data collected from a user or consumer that includes demographic data such as race, age, sex, weight, height, medical conditions, consumption method, and time of consumption. However, other alternatives are contemplated.

The processes 204 may comprise biological data extraction 214, machine learning databases 216, and data mining 218. The process 214 may use the data from inputs 202 described herein, or from alternative sources. The biological data extraction 214 process may extract biological data from the input 202, wherein the data may include the presence or absence biomarkers contained in the metabolomics databases, and ratios of biomarkers compared to other biomarkers detected in the metabolomics data. However, other alternatives are contemplated. The biological data extraction 214 process may also extract data from the cultivar genetic markers input such as genetic sequences, presence or absence of enzymes or other chemicals. The machine learning database 216 may comprise algorithms for performing regression analysis, classification, cluster analysis, feature extraction, correlation analysis, recurrent neural network processing, or deep learning processing. However, other alternatives are contemplated. The data mining 218 processes may comprise tools, libraries, or algorithms for processing the data to extract useful data points. The data mining processes may include feature extraction, cleaning and preparing data for analysis, and presenting data in a visual format such as graphs. However, other alternatives are contemplated.

The outputs 206 generated from the processes may comprise data on biomarkers 220. The biomarker data 220 may comprise data indicating the positive presence or negative presence of a biomarker. Alternatively, the biomarker data 220 may indicate the positive increase or negative decrease of a biomarker compared to other samples such as a control sample. Additionally, the biomarker data 220 may comprise ratios representing the level of one biomarker compared to the level of one or more different biomarkers. However, other alternatives are contemplated.

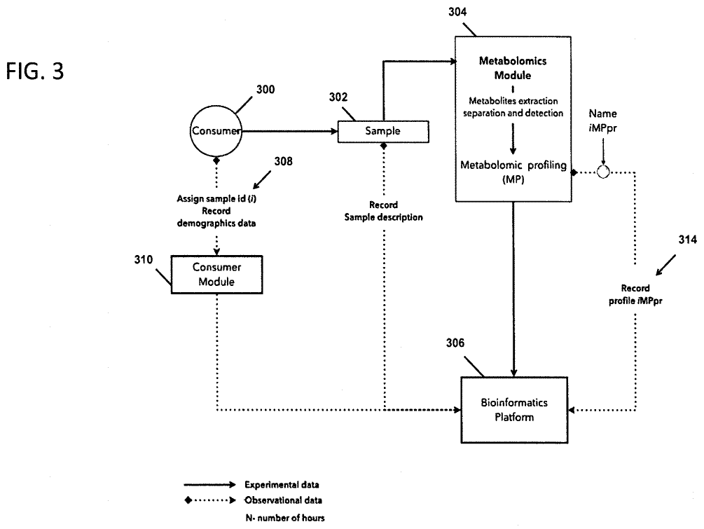

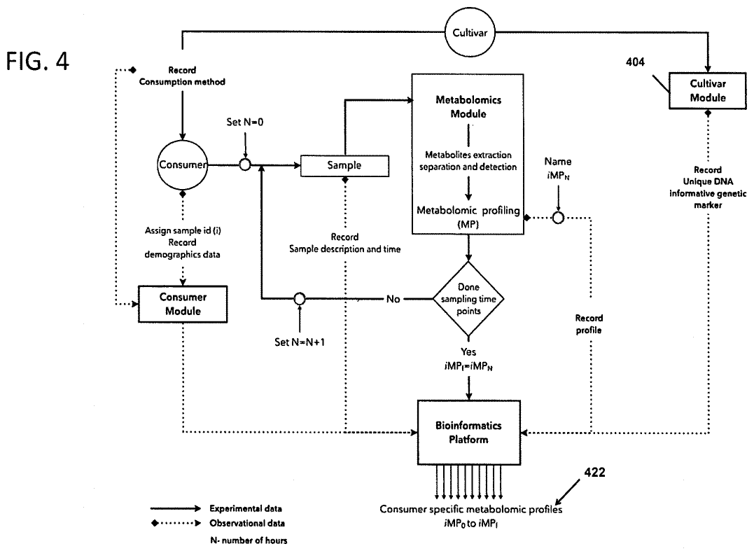

FIGS. 3 and 4 illustrate a schematic presentation of the bioinformatics platform 306 processing overview of consumer 300 sample 302 pre-consumption of intoxicants. Input data from the metabolomics module 304, consumer module 310, cultivar module 404 and the output 422, biomarkers, are indicated. Samples from each consumer prior to cannabis consumption (iMPpr) 314, immediately after consumption (iMPt0) and at indicated time points post consumption (number represent hours; e.g: iMPt2) are subjected to comprehensive metabolomic analysis for profiling metabolites in the metabolomics module and the resultant metabolic profiles (iMPs) processed in the bioinformatics platform. Consumer unique sample id (i) 308 is indicated.

FIG. 4 illustrates a processing overview of consumer samples post-intoxicant consumption. In addition to the process described above in FIG. 3, the processes illustrated in FIG. 4 further includes the collection of multiple samples at multiple time points until complete. Once the sampling is complete, and the metabolomics module has created a metabolomic profile, the metabolomic profile is sent to the bioinformatics platform. Additionally, data from the cultivar module, including recorded unique DNA and informative genetic markers are sent to the bioinformatics platform. According to some examples, the bioinformatics platform may process the data received from the cultivar module, consumer module, and metabolomics module. In some examples, the bioinformatics platform may also receive data from a use module and process that data. In other examples, the bioinformatics platform may use machine learning, artificial intelligence programs, or deep learning to process the data received from other modules.

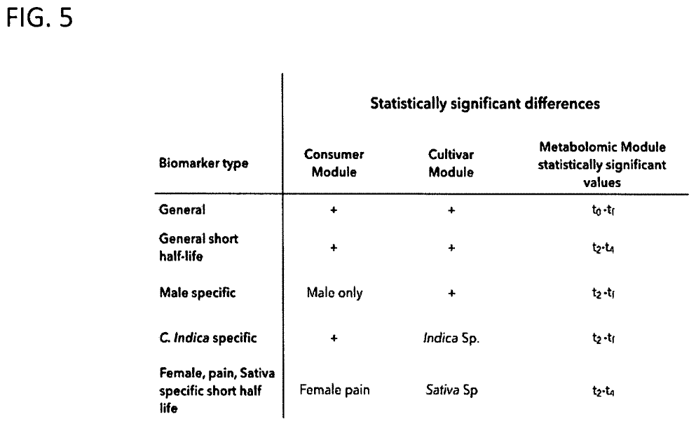

FIG. 5 generally depicts a table representing metabolomics data produced from the processes. The data may later be used for time relevant drug detection such as determining the last time a consumer may have consumed a cultivar or other intoxicant. In other embodiments, the data may be used to recommend cultivars or filter smoke or oil from a cultivar to provide a consumer with a desired affect such as pain relief, anxiety relief, sedation, depression relief, migraine relief, seizure relief, etc.

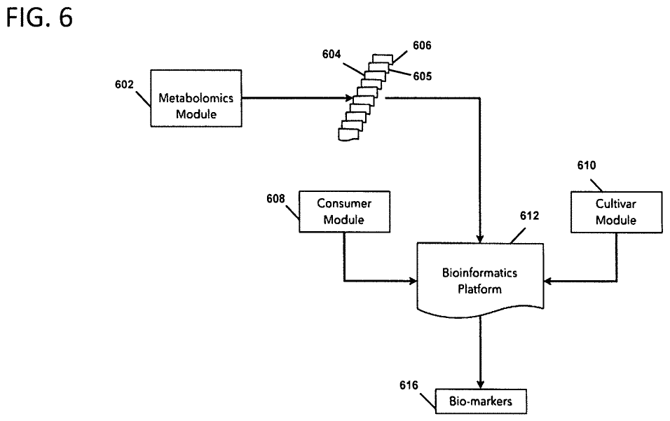

FIG. 6 generally depicts a system for identifying time relevant or pharmaceutical relevant biomarkers, comprising a metabolomics module 602, a metabolomic profile (iMP) at a time point pre consumption 606 of a substance or cultivar and at one or more time points post consumption 604 including during consumption 605, a consumer module 608 which may include a user module mentioned herein, a cultivar module 610, a bioinformatics platform 612, and biomarkers 616.

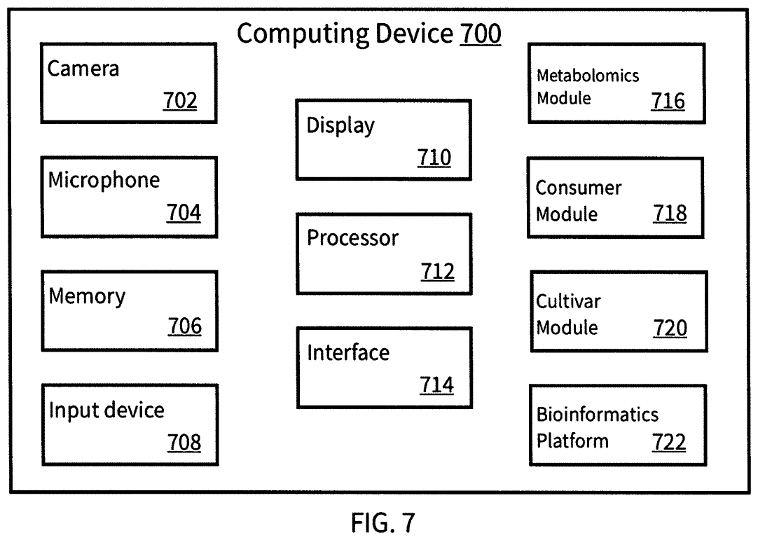

FIG. 7 is a block diagram of an example computing device 700 that can detect biomarkers or ratios of biomarkers that may indicate a time since a cultivar or other intoxicant has been consumed. Alternatively, the detected biomarkers or ratios of biomarkers may indicate a physiological affect that was induced by the consumption of the cultivar or an intoxicant. For example, physiological affects may include anxiety reduction, pain reduction, reduced depression, reduced mental illness symptoms, and increased ability to sleep. The computing device 700 contains a variety of constituent parts and modules that may be implemented through appropriate combinations of hardware, firmware, and software that allow the computing device 700 to function as an embodiment of appropriate features.

The computing device 700 contains one or more processors 712 that may include various hardware devices designed to process data. The processors 712 are communicatively coupled to other parts of computing device 700. For example, the processors 712 may be coupled to a camera 702 and a microphone 704 that allow input of visual and audio signals, respectively, from an area that physically surrounds the computing device 700. The camera 702 and the microphone 704 may provide the raw signals that are processed in other portions of the computing device 700.

The computing device 700 may include a memory 706. The memory 706 may include a variety of memory storage devices, such as persistent storage devices that allow permanent retention and storage of information manipulated by the processors 712.

An input device 708 allows the receipt of commands by the computing device 700 from a user, and an interface 714 allows computing device 700 to interact with other devices to allow the exchange of data. The processors 712 may be communicatively coupled to a display 710 that provides a graphical representation of information processed by the computing device 700 for the presentation to a user.

The processors 712 may be communicatively coupled to a series of modules that perform the functionalities necessary to implement the method of embodiments that are presented in FIG. 6. These modules include an metabolomics module 716, a consumer module 718, a cultivar module 720, and a bioinformatics platform #722.

The method for generating biomarkers consists of 3 modules: 1) Consumer Module; 2) Cultivar Module, and; 3) Metabolomics module. The Modules interface with the bioinformatics platform, which uses machine learning, data mining, pattern recognition, in-house and public databases, statistics and other tools as described with reference to FIG. 2.

The identified biomarkers are measurable substances that indicate a response to cannabis consumption or the presence of molecules originated by the cannabis. A biomarker can indicate the consumption of cannabis, the physiological effect of cannabis consumption on a specific gender or medical condition, the physiological effect of a specific cultivar, or other phenomena.

### Metabolomics Module

Metabolomics refers to the measurement of the metabolite pool that exists within a sample under a particular set of conditions (Fiehn, 2002), while untargeted metabolomics analysis is a technology that allows quantitative measurement of the dynamic multi-parametric metabolic response of living systems to changes such as physiological stimuli or genetic modification.

Metabolites that exhibit quantitative changes that strongly (statistically) correlate with changes in the living systems as a response to physiological stimuli or other changes identify as biomarkers.

In the Metabolomics module, samples are collected from different consumers in a cannabis study with multiple time points that include pre-consumption and post consumption. Protocols for sample generation and collection are designed with no or minimal effect from factors such as diet, exercise, physical activity and time of the day.

The collected samples are stored at −80° C. prior to the data acquisition step. The Metabolomics module consists of two analytical methods which may be performed separately or in combination: 1) Proton nuclear magnetic resonance (1H NMR) (Reo, 2002; Holmes et al. 2006), and 2) mass spectrometry usually coupled with a separation technique such as liquid chromatography (LC) or gas chromatography (GC) (LC/MS and GC/MS respectively) (Plumb et al. 2005; Lindon et al. 2005).

High-resolution NMR techniques has the capacity to detect thousands of metabolites in a wide spectrum of biological objects, including various biological fluids, which are often complex mixtures of organic and inorganic components (Reo, 2002). Since 1H-NMR spectrum contains the well-resolved signals from almost all physiologically significant components, this method can analyze samples for the presence of organic acids, amino acids, protein components, lipids, sugars, nitrogen-containing substances, and others in a short period of time. The relative concentration of components can be calculated accurately from the spectrum.

MS is a very sensitive detection method for metabolites at the picogram level with a dynamic range of three or four orders of magnitude (Lindon et al. 2005). MS detection methods require specific chromatographic methods for the separation and for the ionization matrix and condition that are compatible for components with acidic, basic, amphoteric and neutral characteristics.

NMR and MS separately and in combination on a sample set provide a powerful means of revealing changes in the metabolomic profile. These changes can be assessed in terms of the molecular pathways being perturbed, and allow for the elucidation of the mechanism(s) at work under a particular set of conditions. The ability to link metabolites using these very different analytical techniques increases confidence in the identification of potential biomarkers.

To maximize the information extracted from complex spectroscopic data, chemometric analyses are used to: develop statistical pattern recognition models; achieve optimal characterization of the samples; and detect biomarkers from diverse, highly-dimensional datasets (Deming, 1986; Lavine and Workman, 2008).

In the cases of NMR- and MS-based metabolomics, the chemometric analyses tools involved to process spectra include functions such as peak alignment, peak detection and normalization. In some cases internal standards can be added to the sample matrices to account and normalize for m/z for MS and retention times for GC or LC, and for chemical shifts for NMR. Processed MS-based and NMR-based metabolomics data sets have been described by Crockford et al. (2005), Dieterle et al. (2006), Sysi-Aho et al. (2007) and Warrack et al. (2009),

Statistical analyses may be performed as described below:

Principal component analysis (PCA): an unsupervised pattern recognition technique for classifying sample groups based on the inherent similarity or dissimilarity of their corresponding biochemical compositions.

Partial least-square discriminant analysis (PLS-DA) and orthogonal signal correction (OSC) with partial least squares are supervised methods that can be tested with external test sets or internal cross-validated tests. These external and internal supervised methods can produce less spurious biomarkers than unsupervised PCA methods. Both unsupervised and supervised statistical methods are very useful for pattern recognition after condensing variable dimensions through the combination of all features in sets of NMR or MS spectra.

For useful comparisons of trajectories obtained from different studies, a procedure that permits direct comparison of trajectories, which is referred to as scaled-to-maximum, aligned and reduced trajectories (SMART) can be performed. SMART analysis can also remove inter-laboratory, physiological and phenotypical variations, to correlate dose-response data, and to connect the toxic response in different species or strains of the same species.

Open sources extraction tools such as XCMS (Want et al. 2006), OpenMS (Sturm et al. 2008) and apLCMS (Yu et al. 2009) and databases of metabolites such as Human Metabolome Database (Wishart et al. 2007), METLIN (Smith et al. 2005) and PubChem (Austin et al. 2004) are available for integration with the input of the Consumer, Cultivar and Metabolomics modules. Information from known interactions and pathways can be further used to identify proteins and gene targets that response to physiological effect or other phenomena post cannabis consumption.

### Cultivar Module

Cannabis is originally a naturally occurring weed species that was bred and cultivated to give subtypes with large genetic variation (Coyle, et al. 2003). It can be seed-propagated to create unique genotypes, or perpetuated through cloning to obtain genetic variability via somaclonal variation (Lata, et al. 2011).

Methods for the identification of cannabis plants include: botanical identification through inspection of the intact plant morphology and growth habit, microscopical examination of leaves for the presence of cystolith hairs, chemical screening tests such as the Duquenois-Levine test (Bailey, K. 1979), THC identification through biochemical methods (Hazekamp et al. 2016), and the use of molecular sequencing to identify DNA sequence homology to reference cannabis samples (Coyle, et al. 2003).

Methods for the classification of cannabis cultivars can be performed by DNA testing of mitochondrial, chloroplast and nuclear sequences. DNA-based tests for the individualization of cannabis samples include: 1) Random Amplified Polymorphic DNA (RAPD); Amplified Fragment Length Polymorphism (AFLP); and Short Tandem Repeat (STR).

RADP markers are generated in a single standard PCR reaction where the PCR primers consist of random sequences (typically oligomers of 10-15 bases in length). PCR primers with sequence homology to the DNA template will generate products with variable sizes upon separation on agarose gel and staining, and a unique band pattern can be identified for each cultivar. No prior knowledge of the cultivar sequence is required to perform RADP analysis.

AFLP is widely used to distinguish between individuals of many species such as plants, insects, birds, fish and bacteria. AFLP markers are useful for separating closely-related cultivars from inbred genetic lines in any single source sample. AFLP analysis requires PCR amplification of the DNA template, generated by restriction enzymes digestion, to which adaptor oligomer sequences have been attached. The PCR products detected by the DNA sequencer consist of different sized DNA fragments that form a unique band pattern for each cultivar.

STR sequences refer to repetitive elements found within nuclear DNA that are variable between individuals. The variability in the number of repeated sequences makes these elements useful for distinguishing between individuals in a population. STR analysis requires a PCR reaction using PCR primers of a specific sequence that will bind and recognize a previously-characterized site within the nuclear DNA. Polymorphic loci and microsatellite loci (a tract of repetitive DNA in which certain DNA motifs are repeated), successfully generate diversity patterns among cannabis cultivars (Dufresnes et al. 2017).

In addition to the DNA information, the cultivar module includes information such as: 1)

chemovar (secondary metabolites profile) of: a) medical—cannabinoid content and distribution, b) aroma and/or flavors—terpene content and distribution, and c) water content; 2) Phenotype such as color, consistency, stems, seeds and photos; 3) commercial name; 4) lineage; and 5) known effects such as happy, uplifted, focused giggly and euphoric; etc.

### Consumer Module

The consumer module contains input from: 1) participant data; 2) experimental design; and 3) procedures.

### Participant Data Also Described as a User Profile

Participant data input is extracted from the inclusion criteria questionnaire and includes information such as: medical conditions for cannabis treatment, consumption method, employment, etc.; and demographic information such as: gender, race, and age.

Exclusion criteria include: dependence on other drugs, alcohol dependence, bipolar disorder or schizophrenia, major depressive disorder, suicidal ideation, psychotic symptoms or violent thoughts, current treatment with antidepressant medication, physical illness explaining depressive symptoms (e.g. hypothyroidism, neurological disease, severe anemia, renal failure, etc.), and serious medical conditions that might affect participant safety (eg, cardiac or pulmonary disease, hepatic, or renal disease) or were pregnancy. The database will not positively correlate these conditions with biomarkers.

### Experimental Design

Medical marijuana card holders who sign the informed consent form (ICF) are subjected to the experimental design, which may include crossover design with randomized sequence of study cannabis administration. Participants are assigned to vaporizer containing cannabis or placebo/blank.

For baseline metabolomics data, all selected participants will test negative for cannabinoids, as determined by saliva or other tests, prior to data entry.

### Procedure

Baseline measures based on physical characteristics such as weight, heart rate, etc. will be performed prior to placebo/control treatment for all participants. Participants are blindly randomized to either active or placebo/control treatment, and provided first trial sample (iMPpr).

All participants are treated, for example, daily blindly for a total of 1 week, followed by evaluation of treatment effectiveness. The daily treatment includes sampling pre-treatment and at, for example, 2-3 hours intervals post treatment.

The statistical analysis of questionnaire ratings is performed for example using repeated measure analysis of variance (ANOVA). Comparison of demographic variables is done for example using simple comparisons using paired t-test.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program include, by way of example, may be based on general or special purpose microprocessors or both, or any other kind of central processing unit including graphics processing units. Generally, a central processing unit will receive instructions and data from a read only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer may be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory may be supplemented by, or incorporated in, special purpose logic circuitry.

To provide for interaction with a user, embodiments of the subject matter described in this specification may be implemented on a computer having a display device, e.g., a CRT (cathode ray tube) or LCD (liquid crystal display) monitor, for displaying information to the user and a keyboard and a pointing device, e.g., a mouse or a trackball, by which the user may provide input to the computer. Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, or tactile input. In addition, a computer may interact with a user by sending documents to and receiving documents from a device that is used by the user; for example, by sending web pages to a web browser on a user's client device in response to requests received from the web browser.

Embodiments of the subject matter described in this specification may be implemented in a computing system that includes a back end component, e.g., as a data server, or that includes a middleware component, e.g., an application server, or that includes a front end component, e.g., a client computer having a graphical user interface or a Web browser through which a user may interact with an implementation of the subject matter described in this specification, or any combination of one or more such back end, middleware, or front end components. The components of the system may be interconnected by any form or medium of digital data communication, e.g., a communication network. Examples of communication networks include a local area network (“LAN”) and a wide area network (“WAN”), e.g., the Internet.

The computing system may include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

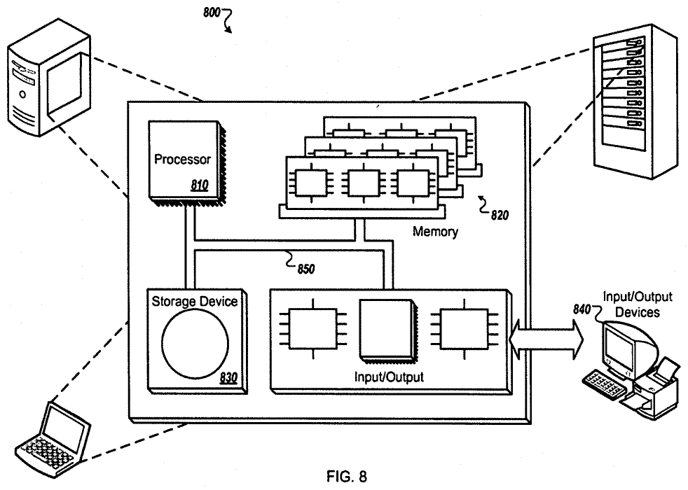

An example of one such type of computer is shown in FIG. 8, which shows a schematic diagram of a generic computer system 800. The system 800 can be used for the operations described in association with any of the computer-implement methods described previously, according to one implementation. The system 800 includes a processor 810, a memory 820, a storage device 830, and an input/output device 840. Each of the components 810, 820, 830, and 840 are interconnected using a system bus 850. The processor 810 is capable of processing instructions for execution within the system 800. In one implementation, the processor 810 is a single-threaded processor. In another implementation, the processor 810 is a multi-threaded processor. The processor 810 is capable of processing instructions stored in the memory 820 or on the storage device 830 to display graphical information for a user interface on the input/output device 840.

The memory 820 stores information within the system 800. In one implementation, the memory 820 is a computer-readable medium. In one implementation, the memory 820 is a volatile memory unit. In another implementation, the memory 820 is a non-volatile memory unit.

The storage device 830 is capable of providing mass storage for the system 800. In one implementation, the storage device 830 is a computer-readable medium. In various different implementations, the storage device 830 may be a floppy disk device, a hard disk device, an optical disk device, or a tape device.

The input/output device 840 provides input/output operations for the system 800. In one implementation, the input/output device 840 includes a keyboard and/or pointing device. In another implementation, the input/output device 840 includes a display unit for displaying graphical user interfaces.

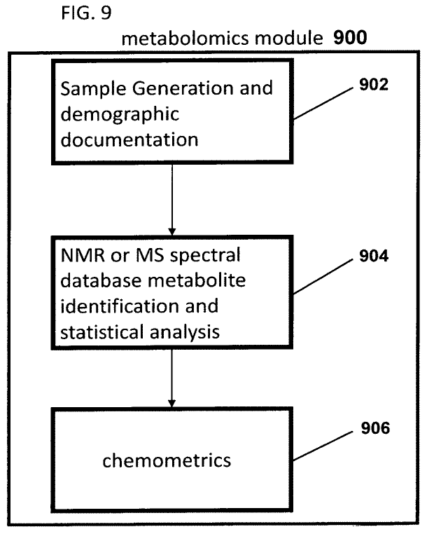

FIG. 9 illustrates a flow chart for Metabolomics module 900 flow chart. The first step 902 is sample generation and demographic documentation. The second step 904 involves data acquisition by untargeted metabolomics approach that uses NMR or MS spectral databases to identify as many metabolites as possible in the analyzed samples, and then use statistics to determine new biomarkers of health or disease status. The third step 906 is chemometrics (identification of spectral or metabolic patterns) data mining for biomarkers. Principal component analysis (PCA) is applied to the NMR or MS data initially to look for patterns and outliers, and to determine if there are any easily discernable biomarkers. After PCA, many other types of supervised methods like partial least squares-discriminate analysis (PLS-DA) and artificial neural networks (ANN) can be employed for further data mining. These may be used to formulate a relationship between metabolomic data and other data types including other omics or imaging endpoints or more classical analyses such as clinical chemistry or histopathology results.

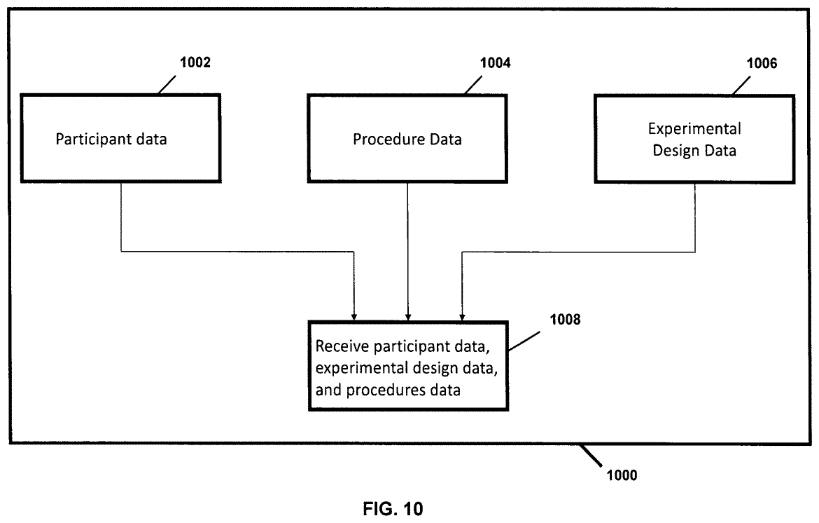

FIG. 10 illustrates a flow chart for a consumer module (also referred to as a user profile) 1000. In one embodiment, the consumer module 1000 may receive and store 1008 input from: 1) participant data 1002; 2) experimental design 1006; and 3) procedures 1004.

### Participant Data 1002

Participant data input is extracted from the inclusion criteria questionnaire and includes information such as: medical conditions for cannabis treatment, consumption method, employment, etc.; and demographic information such as: gender, race, and age.

Exclusion criteria include: dependence on other drugs, alcohol dependence, bipolar disorder or schizophrenia, major depressive disorder, suicidal ideation, psychotic symptoms or violent thoughts, current treatment with antidepressant medication, physical illness explaining depressive symptoms (e.g. hypothyroidism, neurological disease, severe anemia, renal failure, etc.), and serious medical conditions that might affect participant safety (eg, cardiac or pulmonary disease, hepatic, or renal disease) or were pregnancy. The database will not positively correlate these conditions with biomarkers.

### Experimental Design Data 1006

Medical marijuana card holders who sign the informed consent form (ICF) are subjected to the experimental design, which may include crossover design with randomized sequence of study cannabis administration. Participants are assigned to vaporizer containing cannabis or placebo/blank.

For baseline metabolomics data, all selected participants will test negative for cannabinoids, as determined by saliva or other tests, prior to data entry.

### Procedure Data 1004

Baseline measures based on physical characteristics such as weight, heart rate, etc. will be performed prior to placebo treatment for all participants. Participants are blindly randomized to either active or placebo/control treatment, and provided first trial sample (iMPpr).

All participants are treated, for example, daily blindly for a total of 1 week, followed by evaluation of treatment effectiveness. The daily treatment includes sampling pre-treatment and at, for example, 2-3 hours intervals post treatment.

The statistical analysis of questionnaire ratings is performed for example using repeated measure analysis of variance (ANOVA). Comparison of demographic variables is done for example using simple comparisons using paired t-test.

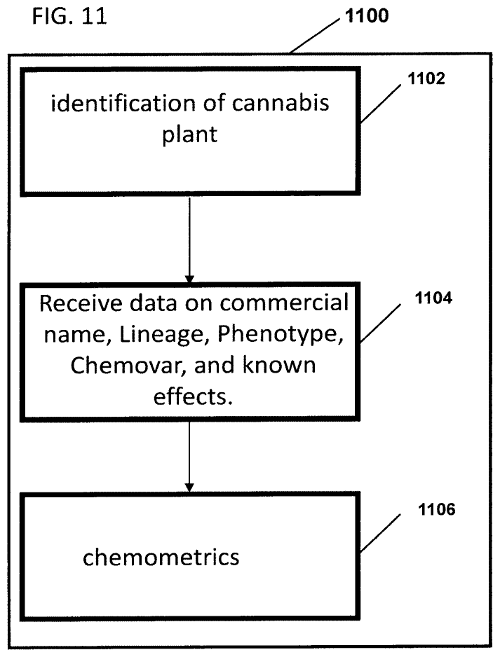

FIG. 11 illustrates a cultivar module 1100 flow chart comprising a step for identification of a cannabis plant 1002 and a step for receiving data on the cannabis plant that may comprise one or more commercial names, lineage, phenotype, chemovar, and known effects 1104. The third step 1106 is chemometrics (identification of spectral or metabolic patterns) data mining for biomarkers. Principal component analysis (PCA) is applied to the NMR or MS data initially to look for patterns and outliers, and to determine if there are any easily discernable biomarkers. After PCA, many other types of supervised methods like partial least squares-discriminate analysis (PLS-DA) and artificial neural networks (ANN) can be employed for further data mining. These may be used to formulate a relationship between metabolomic data and other data types including other omics or imaging endpoints or more classical analyses such as clinical chemistry or histopathology results.

Cannabis is originally a naturally occurring weed species that was bred and cultivated to give subtypes with large genetic variation (Coyle, et al. 2003). It can be seed-propagated to create unique genotypes, or perpetuated through cloning to obtain genetic variability via somaclonal variation (Lata, et al. 2011).

Methods for the identification of cannabis plants include: botanical identification through inspection of the intact plant morphology and growth habit, microscopical examination of leaves for the presence of cystolith hairs, chemical screening tests such as the Duquenois-Levine test (Bailey, K. 1979), THC identification through biochemical methods (Hazekamp et al. 2016), and the use of molecular sequencing to identify DNA sequence homology to reference cannabis samples (Coyle, et al. 2003).

Methods for the classification of cannabis cultivars can be performed by DNA testing of mitochondrial, chloroplast and nuclear sequences. DNA-based tests for the individualization of cannabis samples include: 1) Random Amplified Polymorphic DNA (RAPD); Amplified Fragment Length Polymorphism (AFLP); and Short Tandem Repeat (STR).

RADP markers are generated in a single standard PCR reaction where the PCR primers consist of random sequences (typically oligomers of 10-15 bases in length). PCR primers with sequence homology to the DNA template will generate products with variable sizes upon separation on agarose gel and staining, and a unique band pattern can be identified for each cultivar. No prior knowledge of the cultivar sequence is required to perform RADP analysis.

AFLP is widely used to distinguish between individuals of many species such as plants, insects, birds, fish and bacteria. AFLP markers are useful for separating closely-related cultivars from inbred genetic lines in any single source sample. AFLP analysis requires PCR amplification of the DNA template, generated by restriction enzymes digestion, to which adaptor oligomer sequences have been attached. The PCR products detected by the DNA sequencer consist of different sized DNA fragments that form a unique band pattern for each cultivar.

STR sequences refer to repetitive elements found within nuclear DNA that are variable between individuals. The variability in the number of repeated sequences makes these elements useful for distinguishing between individuals in a population. STR analysis requires a PCR reaction using PCR primers of a specific sequence that will bind and recognize a previously-characterized site within the nuclear DNA. Polymorphic loci and microsatellite loci (a tract of repetitive DNA in which certain DNA motifs are repeated), successfully generate diversity patterns among cannabis cultivars (Dufresnes et al. 2017).

In addition to the DNA information, the cultivar module includes information such as: 1) chemovar (secondary metabolites profile) of: a) medical—cannabinoid content and distribution, b) aroma and/or flavors—terpene content and distribution, and c) water content; 2) Phenotype such as color, consistency, stems, seeds and photos; 3) commercial name; 4) lineage; and 5) known effects such as happy, uplifted, focused giggly and euphoric; etc.

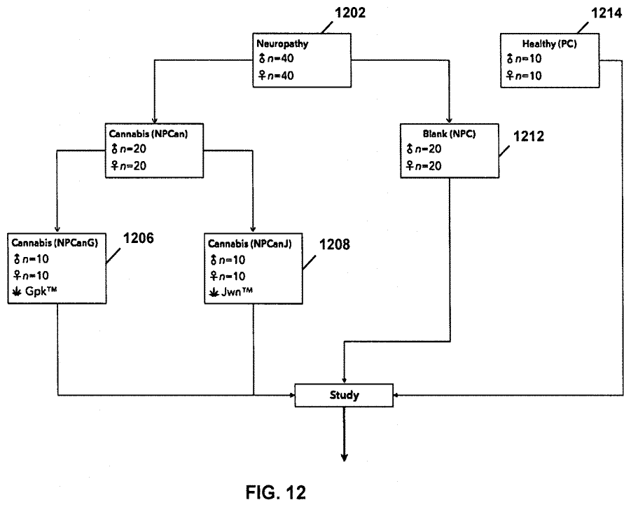

FIG. 12 Flow diagram of neuropathy patients 1202 trial study. The number of neuropathy patients consumed cannabis cultivars Gpk™ 1206 and Jwn™ 1208 (NPCanG and NPCanJ respectively) and control group consumed blank (NPC) 1212 are indicated. Healthy participants 1214 served as positive control (PC).

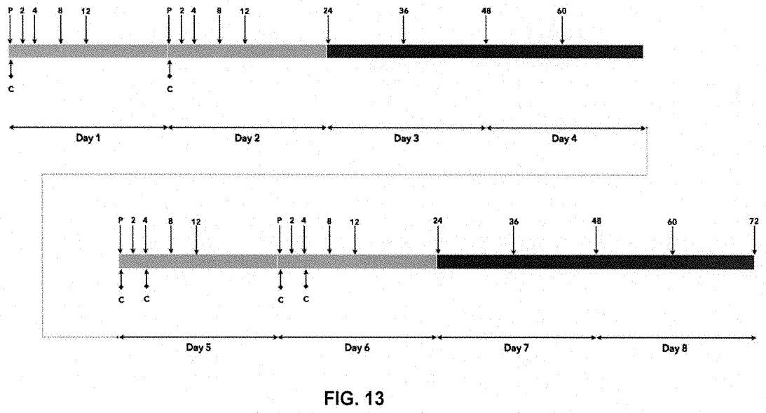

FIG. 13 Illustration of time series data from trial study. More specifically, data shown is of cannabis or blank consumption (C) and time of sampling and pain assessment in hours relative to consumption to the first consumption of the day. P indicates time pre-consumption (10 minute prior inhaling cannabis or blank). Consumption (light gray) and wash-out (dark gray) days are indicted.

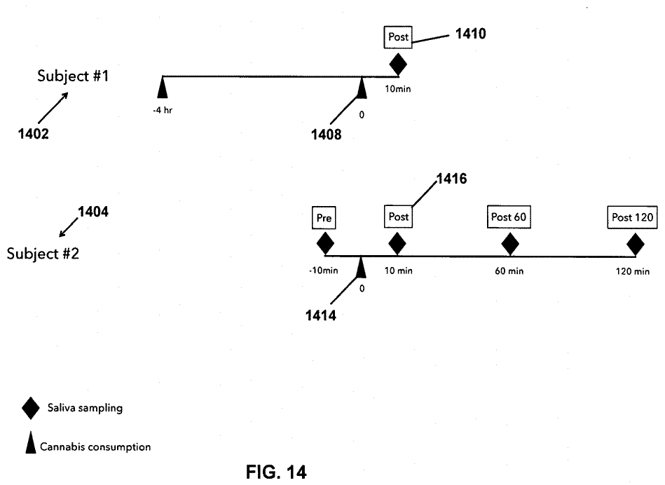

FIG. 14 Illustration of subject #1 1402 and subject #2 1404 consumption and sampling schedule. Cannabis consumption and sampling are indicated by triangle and diamond respectively. Time 0 1408 and 1414 were set to the last cannabis consumption prior to first post consumption sampling (10 min) 1410 and 1416.

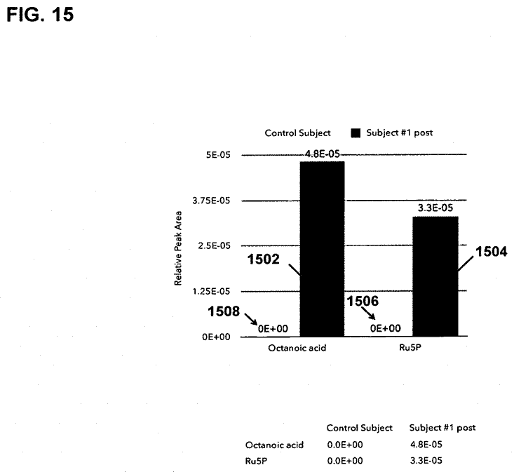

FIG. 15 the effect of cannabis consumption on the levels of Octanoic acid and Ribulose 5-phosphate (Ru5P) detected in cannabis free subject (Control Subject) and Subject #1 10-min post cannabis consumption (Subject #1 Post). Octanoic acid and Ru5P relative area peak area are indicated below the graphs. Subjects characteristics and consumption details described in tables 1 and 2 respectively. The chart indicates the control subjects octanoic acid levels 1508 are Ru5P levels 1506. Additionally, the chart indicates chart indicates the Subject #1 Post subjects octanoic acid levels 1502 are Ru5P levels 1504.

Additionally, FIG. 15 describes examples for metabolic cannabis consumption biomarkers that are consumption specific including but not limited to Octanoic acid and Ribulose 5-phosphate (Ru5P) that are absent in cannabis free subject (Subject Control) and present in Subject #1 10-min post-consumption (Subject #1 Post).

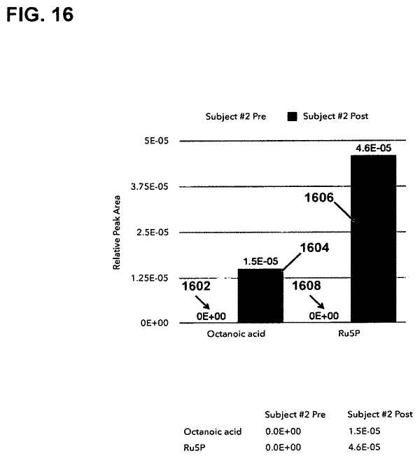

FIG. 16 describes examples for metabolic cannabis consumption biomarkers that are consumption specific including but not limited to Octanoic acid and Ribulose 5-phosphate (Ru5P) that isabsent in Subject #2 prior to cannabis consumption (Subject #2 Pre) and present 10 min post cannabis consumption (Subject #2 Post). The chart indicates the Subject #2 Pre octanoic acid levels 1602 are Ru5P levels 1608. Additionally, the chart indicates chart indicates the Subject #2 Post octanoic acid levels 1604 are Ru5P levels 1606.

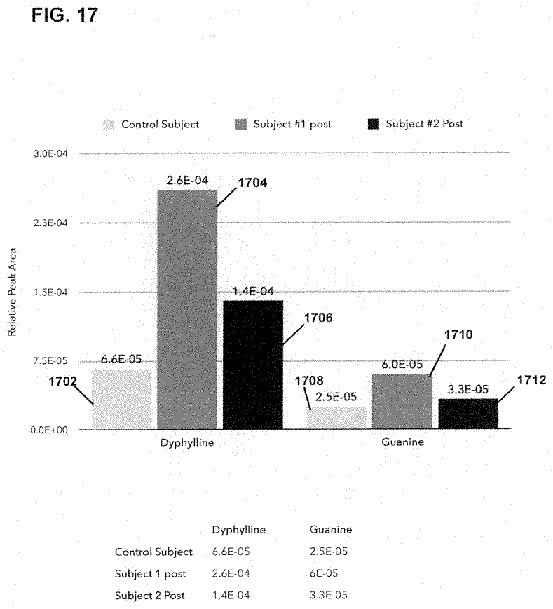

FIG. 17 illustrates cannabis consumption biomarkers which affect metabolic pathways. These are THC-level dependent and include but are not limited to Dyphylline and guanine. These exhibit: (0) low levels in saliva obtained from the Control subject for Dyphylline 1702 and Guanine 1708; and (1) high levels in saliva obtained from Subject #1 in Dyphylline 1704 and Guanine 1710, who consumed cannabis 250 minutes (THC 27%) and 10 minutes (THC 23.5%) prior to sampling ; and (2) high levels in saliva from Subject #2 in Dyphylline 1706 and Guanine 1712, who consumed cannabis 10 minutes (THC 9.8%) prior to sampling Significantly higher levels of guanine (p<0.0031) were identified in 18 cocaine-dependent subjects and ten controls who had participated in a cocaine addiction study (Patkar., et al. 2009). The study indicated that blood drawn from cocaine addicted subjects exhibit oxidative stress through generation of reactive oxygen species and alteration of the purine (adenosine and guanine) pathways.

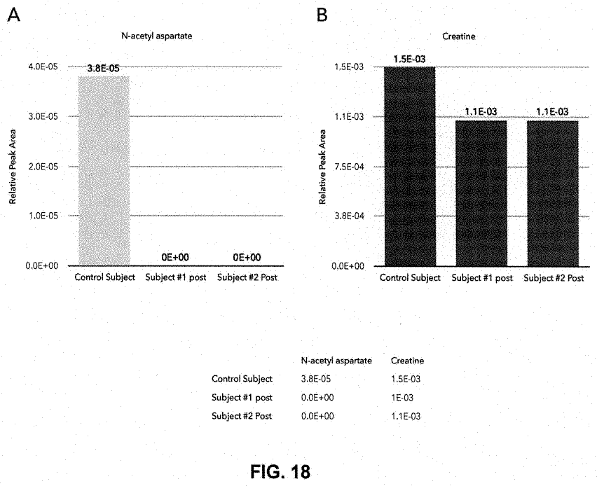

FIG. 18 Illustrates cannabis consumption biomarkers which affect metabolic pathways. These biomarkers show a decrease post-consumption, including but not limited to, N-acetyl aspartate (16A) and creatine (16B). Prescot et al., (2011) demonstrated using a non-invasive Proton (1H) Magnetic Resonance Spectroscopy (MRS) that 17 adolescent marijuana users showed statistically significant reductions in N-acetyl aspartate and total creatine levels compared to similarly aged healthy control subjects. FIG. 18 further illustrates the effect of cannabis consumption on the levels of N-acetyl aspartate (A) and creatine (B) detected in cannabis free subject (Control Subject), Subject #1 10-min post cannabis consumption (Subject #1 Post) and Subject #2 10-min post cannabis consumption (Subject #2 Post). N-acetyl aspartate and creatine relative area peaks area are indicated below the graphs.

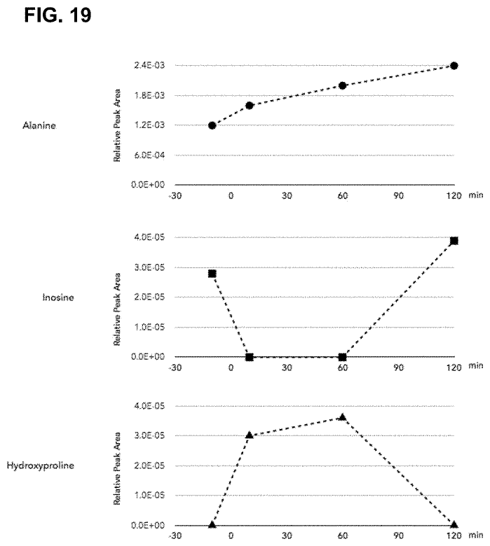

FIG. 19 describes examples for cannabis consumption biomarkers which affect metabolic pathways. These biomarkers are time-dependent, including but not limited to alanine, inosine and hydroxyproline.

According to some example embodiments, means for detecting untargeted biomarkers are described. Example means for detecting untargeted biomarkers may include means for receiving, by the one or more computing devices, a genetic and chemical profile of a cultivar; a means for receiving, by the one or more computing devices, a user profile, wherein the user profile may comprises method of consumption and demographic data of a consumer; and a means for receiving, by the one or more computing devices, a metabolomic profile of said consumer, wherein the metabolomic profile comprises metabolomics data acquired at a time prior to consumption of said cultivar, at a time of consumption of said cultivar, and at a time after the consumption of said cultivar. Example means for detecting untargeted biomarkers may further include means for generating, by the one or more computing devices, from said comparing step, a differences profile comprising differences recognized by the one or more computing devices in said metabolomics data acquired at said time prior to consumption of said cultivar to said metabolomics data acquired at said time of consumption of said cultivar, and said metabolomics data acquired at said time after the consumption of said cultivar; and means for calculating, by the one or more computing devices, a correlation between said time of consumption of said cultivar and one or more data points from said genetic and chemical profile of said cultivar, said user profile, or said metabolomics data of said differences profile.

In one embodiment, a method for detecting untargeted biomarkers is described. Example methods may include receiving, by the one or more computing devices, a genetic and chemical profile of a cultivar; receiving, by the one or more computing devices, a user profile, wherein the user profile may comprise method of consumption and demographic data of a consumer; receiving, by the one or more computing devices, a metabolomic profile of said consumer, wherein the metabolomic profile comprises metabolomics data acquired at a time prior to consumption of said cultivar, at a time of consumption of said cultivar, and at a time after the consumption of said cultivar; and comparing, by the one or more computing devices, said metabolomics data acquired at a time prior to consumption of said cultivar to said metabolomics data acquired at a time of consumption of said cultivar, and said metabolomics data acquired at a time after the consumption of said cultivar. Example methods for detecting untargeted biomarkers may further include generating, by the one or more computing devices, from said comparing step, a differences profile comprising differences recognized by the one or more computing devices in said metabolomics data acquired at said time prior to consumption of said cultivar to said metabolomics data acquired at said time of consumption of said cultivar, and said metabolomics data acquired at said time after the consumption of said cultivar; and calculating, by the one or more computing devices, a correlation between said time of consumption of said cultivar and one or more data points from said genetic and chemical profile of said cultivar, said user profile, or said metabolomics data of said differences profile. The metabolomic profile may include a plurality of ratios of biomarkers within the metabolomic profile.

According to other examples, the calculating step may further comprise training a machine learning algorithm to estimate said time of consumption of said cultivar using said genetic and chemical profile of said cultivar, said user profile, and said metabolomics data of said differences profile; using said machine learning algorithm to estimate an unknown time of consumption of an unknown cultivar using new data comprising a plurality of data points from said user profile, and said metabolomics data of said differences profile; and generating a confidence score confidence score that represents the likelihood that a consumer consumed marijuana or another intoxicant within a specific time frame. The method may further comprise a step of feature extraction.

According to some examples a system comprising one or more computers and one or more storage devices storing instructions that are operable, when executed by the one or more computers, to cause the one or more computers to perform operations for detecting untargeted biomarkers. An example system may include receiving, by the one or more computing devices, a genetic and chemical profile of a cultivar; receiving, by the one or more computing devices, a user profile, wherein the user profile comprises: method of consumption and demographic data of a consumer; receiving, by the one or more computing devices, a metabolomic profile of said consumer, wherein the metabolomic profile comprises metabolomics data acquired at a time prior to consumption of said cultivar, at a time of consumption of said cultivar, and at a time after the consumption of said cultivar; and comparing, by the one or more computing devices, said metabolomics data acquired at a time prior to consumption of said cultivar to said metabolomics data acquired at a time of consumption of said cultivar, and said metabolomics data acquired at a time after the consumption of said cultivar. Example systems for detecting untargeted biomarkers may further include generating, by the one or more computing devices, from said comparing step, a differences profile comprising differences recognized by the one or more computing devices in said metabolomics data acquired at said time prior to consumption of said cultivar to said metabolomics data acquired at said time of consumption of said cultivar, and said metabolomics data acquired at said time after the consumption of said cultivar; and calculating, by the one or more computing devices, a correlation between said time of consumption of said cultivar and one or more data points from said genetic and chemical profile of said cultivar, said user profile, or said metabolomics data of said differences profile. The metabolomic profile may include a plurality of ratios of biomarkers within the metabolomic profile.

According to other examples, the calculating step may comprise training a machine learning algorithm to estimate said time of consumption of said cultivar using said genetic and chemical profile of said cultivar, said user profile, and said metabolomics data of said differences profile; using said machine learning algorithm to estimate an unknown time of consumption of an unknown cultivar using new data comprising a plurality of data points from said user profile, and said metabolomics data of said differences profile; and generating a confidence score confidence score that represents the likelihood that a consumer consumed marijuana or another intoxicant within a specific time frame. The system may further comprise a step of feature extraction

In another embodiment, a non-transitory computer-readable medium storing software comprising instructions executable by one or more computers which, upon such execution, cause the one or more computers to perform operations for detecting untargeted biomarkers. An example non-transitory computer-readable medium may include receiving, by the one or more computing devices, a genetic and chemical profile of a cultivar; receiving, by the one or more computing devices, a user profile, wherein the use profile may comprise a method of consumption and demographic data of a consumer; receiving, by the one or more computing devices, a metabolomic profile of said consumer, wherein the metabolomic profile comprises metabolomics data acquired at a time prior to consumption of said cultivar, at a time of consumption of said cultivar, and at a time after the consumption of said cultivar; comparing, by the one or more computing devices, said metabolomics data acquired at a time prior to consumption of said cultivar to said metabolomics data acquired at a time of consumption of said cultivar, and said metabolomics data acquired at a time after the consumption of said cultivar. Example non-transitory computer-readable medium for detecting untargeted biomarkers may further include generating, by the one or more computing devices, from said comparing step, a differences profile comprising differences recognized by the one or more computing devices in said metabolomics data acquired at said time prior to consumption of said cultivar to said metabolomics data acquired at said time of consumption of said cultivar, and said metabolomics data acquired at said time after the consumption of said cultivar; and calculating, by the one or more computing devices, a correlation between said time of consumption of said cultivar and one or more data points from said genetic and chemical profile of said cultivar, said user profile, or said metabolomics data of said differences profile. The metabolomic profile may include a plurality of ratios of biomarkers within the metabolomic profile.

According to other embodiments, the calculating step may comprise training a machine learning algorithm to estimate said time of consumption of said cultivar using said genetic and chemical profile of said cultivar, said user profile, and said metabolomics data of said differences profile; using said machine learning algorithm to estimate an unknown time of consumption of an unknown cultivar using new data comprising a plurality of data points from said user profile, and said metabolomics data of said differences profile; and generating a confidence score confidence score that represents the likelihood that a consumer consumed marijuana or another intoxicant within a specific time frame. The non-transitory computer-readable storage medium may further comprise a step of feature extraction.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments may also be implemented in combination in a single embodiment. Conversely, various features that are described in the context of a single embodiment may also be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems may generally be integrated together in a single software product or packaged into multiple software products.

Particular embodiments of the subject matter have been described. Other embodiments are within the scope of the following claims. For example, the actions recited in the claims may be performed in a different order and still achieve desirable results. As one example, the processes depicted in the accompanying figures do not necessarily require the particular order shown, or sequential order, to achieve desirable results. In certain implementations, multitasking and parallel processing may be advantageous.

### Example: Metabolomics Approach for Cannabis Biomarkers Discovery

The inclusion criteria were men and women aged 21-55 years with neuropathic pain of at least three months in duration caused by trauma or surgery, with allodynia or hyperalgesia and did not consume cannabis during the last month before the study (negative urine and saliva cannabinoid test).

### Cannabis User

Medical marijuana card holders were recruited at the medical cannabis dispensary collective (MCD) Purple Star, M. D, San Francisco Calif. exhibiting normal liver function (defined as aspartate aminogransferase less than three times normal), normal renal function (defined as a serum creatinine level<133 μmon), normal hematocrit (>38%) and a negative result on β human chorionic gonadotropin pregnancy test (if applicable).

The exclusion criteria were pain due to cancer, current substance abuse or dependence (including abuse of or dependence on cannabis), history of psychotic disorder, current suicidal ideation, pregnancy or breastfeeding, participation in another clinical trial within 30 days of enrollment in our trial, and ongoing insurance claims.

### Control

Subjects were patient's spouses and volunteers who are free of neuropathic pain and did not consume cannabis during the last 3 month before the study (negative urine and saliva cannabinoid test). The suitability of spouses is that by virtue of marital choices and shared experiences, spouses tend to be alike in age, education, socioeconomic status, race religion and environmental factors such as diet and exposure to possible toxins.

### Measures

The following questionnaires were used in this study for diagnostic assessment; demographic questionnaire (name, age, sex, employment, etc.). Baseline measures based on physical characteristics of height, weight and heart rate were recorded prior to the study.

Participants also rated the quality and intensity of their pain experience on the McGill Pain Questionnaire (Chapman et al, 1985; Melzack, 1975). This included the Visual Analog Scale (VAS), a 10-cm line anchored at one end by the descriptor “No Pain” and at the other by the words “Worst Pain Imaginable”.

### Procedures

All 100 participants (neuropathy patients: 80; and control: 20) were required to take part in an induction day, which included an introduction talk, safety training, saliva and cannabis sample collection protocols, vaporizer operation and pain evaluation. Afterwards, neuropathy patients were divided to either active cannabis consumer (NPCan) or control (NPC) (40 participants in each group) FIG. 12. Control group was designated—PC. The demographic characteristics (age and sex) and physical characteristics (height and weight) in the NPCan, NPC and PC groups were distributed similarly.

### Treatment Regime

NPCan group was subdivided to two groups based on the medical cannabis cultivar (NPCanG: consumed cultivar Gpk™ and; NPCanJ: consumed cultivar Jwn™). To reduce non-specific effect on the saliva metabolomics, cultivars with no known dry mouth side effect were selected for the treatment.

As described in FIG. 13, each subject participated in 8 days study that included 4 segments with the following steps: 1) Saliva sampling and pain assessment prior to cannabis (NPCan group) and blank (NPC and PC groups) consumption followed by saliva sampling and pain assessment 2, 4, 8 and 12 hours in days 1 and 2; 2) 2 days wash-out to allow for cannabis decay with saliva sampling and pain assessment every 12 hours; 3) as described in segment 1, with a second consumption session after 4 hours and; 4) as described in segment 2 followed by final sampling and pain assessment 72 hours post cannabis consumption. Eating and drinking foods with high sugar, acidity and caffeine content were not permitted for at least 1 hour prior to saliva collection. Consumption of alcohol, nicotine, and prescription/over-the-counter medications were not permitted during the 8-days study.

### Consumption Session

Vaporization of cannabis was performed according to the instruction supplied for PAX 2 by PAX Labs, San Francisco, Calif. Participants inhaled one cannabis or blank puff per temperature setting (low: 182° C., med-low: 193° C., med-high: 205° C. and high: 215 ° C.), and inhaled an additional one puff per temperature setting 10 minutes later.

### Saliva Samples Collection

Each participants rinsed their mouth with water to remove food residue 10 minutes prior to sampling and approximately 500 μl whole saliva was collected in was collected in pre-labeled 2 ml Cryogenic vial (Nalgene™ Thermo Scientific™, USA) and immediately stored in dry ice. Samples transferred daily to −80 ° C. for storage until the end of the study.

### Metabolomic Analysis of Saliva

### Saliva Samples Preparation

Frozen saliva was thawed at 4° C. for approximately 1.5 hours and subsequently dissolved using a vortex mixer at room temperature and centrifuged through a 5-kDa cutoff filter (Millipore, Bedford, Mass.) at 9,100×g for at least 2.5 hours at 4° C.; 45 μl of each sample was added to a 1.5 ml Eppendorf tube, with 2 mM of methionine sulfone, 2-[N-morpholino]-ethanesulfonic acid (MES), D-Camphol-10-sulfonic acid, sodium salt, 3-aminopyrrolidine, and trimesate.

### Metabolite Standards

All chemical standards were obtained from common commercial sources and dissolved in Milli-Q (Millipore, Bedford, Mass.) water, 0.1 N HCl or 0.1 N NaOH to obtain 10 mM or 100 mM stock solutions. Working standard mixtures were prepared by diluting stock solutions with Milli-Q water just prior to injection into the CE-TOFMS. The chemicals used were of analytical or reagent grade.

### Instrumentation

All CE-TOFMS experiments were performed using an Agilent CE capillary electrophoresis system (Agilent Technologies, Waldbronn, Germany), an Agilent G3250AA LC/MSD TOF system (Agilent Technologies, Palo Alto, Calif.), an Agilent1100 series binary HPLC pump, and the G1603A Agilent CE-MS adapter and G1607A Agilent CE-ESI-MS sprayer kit. For system control and data acquisition we used the G2201AA Agilent ChemStation software for CE and the Analyst QS for Agilent TOFMS software. CE-MS/MS analyses for compound identification were performed on a Q-Star XL Hybrid LC-MS/MS System (Applied Biosystems, Foster City, Calif.) connected to an Agilent CE instrument.

### CE-TOFMS Conditions for Cationic Metabolite Analysis

Separations were carried out in a fused silica capillary filled with 1 M formic acid as the electrolyte. Sample solution were injected at 50 mbar for 3 s, and 30 kV of voltage was applied. The capillary temperature was maintained at 20° C., and the sample tray was cooled below 5° C. Methanol-water (50% v/v) containing 0.5 μM reserpine was delivered as the sheath liquid at 10 μl/min. ESI-TOFMS was operated in the positive ion mode, and the capillary voltage was set at 4,000 V. A flow rate of heated dry nitrogen gas (heater temperature 300° C.) was maintained at 10 psig. In TOFMS, the fragmentor, skimmer, and Oct RFV voltage were set at 75 V, 50 V, and 125 V, respectively. Automatic recalibration of each acquired spectrum was performed using reference masses of reference standards.

### CE-TOFMS Conditions for Anionic Metabolite Analysis

A cationic polymer-coated SMILE (+) capillary was used as the separation capillary. A 50 mM ammonium acetate solution (pH 8.5) was used as electrolyte solution for CE separation. Sample solution was injected at 50 mbar for 30 s and −30 kV of voltage was applied. Ammonium acetate (5 mM) in 50% methanol-water (v/v) containing 20 μM PIPES and 1 μM reserpine was delivered as the sheath liquid at 10 μl/min. ESI-TOFMS was conducted in the negative ion mode; the capillary voltage was set at 3,500 V. For TOFMS, the fragmentor, skimmer, and Oct RFV voltage were set at 100 V, 50 V, and 200 V, respectively. Automatic recalibration of each acquired spectrum was performed using reference masses of standards

### CE-Q-TOFMS Conditions for the Acquisition of MS/MS Spectra

Most of the conditions were identical to those in cationic metabolite analysis using CE-TOFMS. Methanol-water (50% v/v) containing 1 μM reserpine was delivered as the sheath liquid at 5 μl/min. ESI-Q-TOFMS was conducted in the positive product ion scan mode; the ion spray voltage was set at 5,500V.

### Data Processing for Metabolome

Raw datasets were preprocessed by binning the data along the m/z axis to 0.02 m/z resolution, subtracting the baseline from each electropherogram by robust nonlinear fitting of the data to a 7th order polynomial and removing the noise from each electropherogram by leveling to 0 all signal intensity values that fell within 5× S.D. of the signal intensities from 1 to 4 min. The resulting data sets were then further binned to 1 m/z unit resolution along the m/z axis. A set of peaks was selected from each dataset using a modified Douglas-Peucker algorithm. Annotation tables for both cation and anion modes were generated based on the results of the CE-TOFMS analysis of standard compounds. The annotation labels were aligned to the actual datasets in a similar fashion. Arithmetic operations were applied to whole datasets on a data point-by-data point basis to highlight differences of interest.

### Determination of Metabolite Concentrations in Saliva

The concentrations were evaluated by the Mann-Whitney test and P-values for evaluating differences in metabolite concentrations between NPCan and controls were corrected by false discovery rate (FDR) for considering multiple independent tests. For the other parameters, Mann-Whitney and Chi-square tests were used for quantitative and qualitative variables, respectively.

### Cultivar Analysis

### Cultivars

1) Gpk™: The cultivar is an indica-dominant cross between Gog™, Pk™, and Afg™ bred by MTG Seeds California.
2) Jwn™: The cultivar is an Hawaiian sativa-dominant cross between H78™ and Lb™ bred by Pua Mana 1st Hawaiian Pakalōlō Seed Bank.

### Genomic DNA Extraction

Leaf samples from individual plants were collected by participants from the NPCan group and stored at −80° C. Fifty mg of plant material was disrupted in liquid nitrogen using sterile ceramic mortar and pestle and DNA extracted using the plant DNeasy kit (QIAGEN Valencia, Calif.) according to the manufacturer recommendation.

### DNA Amplification

The reaction components for the Random Amplified Polymorphic DNA (RAPDs) method were as follows: 25 ng genomic DNA , 0.2 M 10 bp oligomer primer (Operon Technologies, Alameda, California), 1× Taq DNA polymerase reaction buffer (50 mM CKl, 10 mM Tris HCl (pH 9.0), 0.1% Triton X-100), 2.5 mM MgCl2, 200 M each of dATP, dCTP, dGTP and TTP (Boehringer-Mannheim, Laval, Que.) and 1.25 U Taq DNA polymerase (Gibco BRL, Gaithersburg, Md.). These components were included in a total reaction volume of 25 μl. Amplification was performed in C1000 Touch™ Thermal Cycler (Bio-Rad, Hercules, Calif.) using the following temperature profile: 94° C. for 1.5 min; then 94° C. for 30 sec; 36° C. for 35 sec, 72° C. for 1 min for 45 cycles; then 72° C. for 2 min. The following 10-mers primers utilized for DNA amplification were purchased from Operon Technologies, (Alameda, Calif.): OPA-20 (GTTGCGATCC), OPB-08 (GTCCACACGG), OPG-17 (ACGACCGACA), OPG-19 (GTCAGGGCAA), OPH-05 (AGTCGTCCCC) and OPH-08 (GAAACACCCC).

### Polymorphisms Analysis

Each DNA fragment generated was treated as a separate character and scored as a discrete variable, using 1 to indicate presence and 0 for absence. Accordingly, a rectangular binary data matrix was obtained and statistical analysis was performed using the NTSYS-pc (Rohlf 1992) statistical package. A pairwise similarity matrix was generated using Jaccard's coefficient (Dunn and Everitt 1982) by means of SIMQUAL procedure of NTSYS-pc.

The analysis of molecular variance (AMOVA, Excoffier et al. 1992) was performed using GENALEX 6 (Peakall and Smouse 2006) to partition the total molecular variance between and within populations. significance level was detected via permutation test (n=1,000).

### Example: Characterization of Biomarkers Indicative of Cannabis Consumption

### 1. Samples Collection

To measure cannabis consumption accurately subjects described in table 1 avoided major meal 60 min prior to consumption, rinsed mouth immediately after consumption with water and waited 10 min prior to saliva collection. Saliva samples (0.5 ml-1 ml) were collected using the Saliva Passive Drool Collection Kit (Salimetrics, LLC, Carlsbad, Calif.) and immediately frozen on dry ice.

### Subjects Sampling Description

Control Subject: no cannabis consumption (single time point only)

Subject #1 Post, single time point sampling 10 min post cannabis consumption by vaping.

Subject #2: (Pre) 10 min prior to cannabis consumption by vaping and (post) 10, 60 and 120 min post cannabis consumption by vaping.

### 2. Samples Preparation

Frozen saliva was thawed and dissolved at room temperature. Prior to the metabolome analyses, each saliva sample (50 μL) was mixed with 20 μL of Milli-Q (Merck Millipore, Billerica, Mass., USA) containing internal standards and 20 mM each of methionine sulfone, D-camphor-10-sulfonic acid (Wako Pure Chemical Industries, Ltd. Osaka, Japan), 2-(n-morpholino)ethanesulfonic acid (Dojindo Molecular Technologies, Inc., Kumamoto, Japan), 3-aminopyrrolidine (Sigma-Aldrich Japan K.K., Tokyo, Japan), and trimesate (Wako Pure Chemical Industries, Ltd.) and 30 μL of Milli-Q water. The mixture was then filtrated through 5-kDa cut-off filter (ULTRAFREE-MC-PLHCC, Human Metabolome Technologies, Yamagata, Japan) to remove macromolecules.

### 3. Measurement

The compounds were measured in the Cation and Anion modes of CE-TOFMS (Agilent Technologies, Santa Clara, Calif.) based metabolome as described by Soga et al., 2003.

### Cationic Metabolites (Cation Mode)

Agilent CE-TOFMS system (Agilent Technologies Inc.) Capillary: Fused silica capillary i.d. 50 μm×80 cm

### Analytical Condition

Run buffer: Cation Buffer Solution (p/n: H3301-1001)
Rinse buffer: Cation Buffer Solution (p/n: H3301-1001)
Sample injection: Pressure injection 50 mbar, 10 sec
CE voltage: Positive, 27 kV
MS ionization: ESI Positive
MS capillary voltage: 4,000 V
MS scan range: m/z 50-1,000
Sheath liquid: HMT Sheath Liquid (p/n: H3301-1020)

### Anionic Metabolites (Anion Mode)

Agilent CE-TOFMS system (Agilent Technologies Inc.) Capillary: Fused silica capillary i.d. 50 μm×80 cm

### Analytical Condition

Run buffer: Anion Buffer Solution (p/n: H3302-1021)
Rinse buffer: Anion Buffer Solution (p/n: H3302-1021)
Sample injection: Pressure injection 50 mbar, 25 sec
CE voltage: Positive, 30 kV
MS ionization: ESI Negative
MS capillary voltage: 3,500 V
MS scan range: m/z 50-1,000
Sheath liquid: HMT Sheath Liquid (p/n: H3301-1020)

### 4. Data Processing and Analysis

### 4.1 Data Processing

Peaks detected in CE-TOFMS analysis were extracted using automatic integration software (MasterHands ver. 2.17.1.11 developed at Keio University) in order to obtain peak information including m/z, migration time (MT), and peak area. The peak area was then converted to relative peak area by the following equation described below. The peak detection limit was determined based on signal-noise ratio; S/N=3.

$$Relative Peak Area = \frac{Metabolite Peak Area}{Internal Standard Peak Area \times Sample Amount}$$

### 4.2 Annotation of Peaks

Putative metabolites were then assigned from HMT (Human Metabolome Technologies Inc Tokyo,Japan) standard library and Known-Unknown peak library on the basis of m/z and MT. The tolerance was ±0.5 min in MT and ±10 ppm* in m/z. If several peaks were assigned the same candidate, the candidate was given the branch number.

$${}^{*} Mass error ( ppm ) = \frac{Measured Value - Theoretical Value}{Measured Value} \times {10}^{6}$$

### 4.3 Statistical Analysis (PCA, HCA)

Hierarchical cluster analysis (HCA) and principal component analysis (PCA) were performed by statistical analysis software (developed at HMT). The analysis results were shown in the attached Excel file in detail.

### 4.4 Plotting on Pathway Map

The profile of peaks with putative metabolites are represented on metabolic pathway maps using VANTED (Visualization and Analysis of Networks containing Experimental Data) software. The pathway map was prepared based on the metabolic pathways that are known to exist in human cells according to the information in KEGG database (http://www.genome.jp/kegg/).

TABLE 1
Subjects characteristics

| | Control Subject | Subject #1 | Subject #2 |
|---|---|---|---|
| | Non-cannabis | Cannabis | Cannabis |
| Description | consumer | consumer | consumer |
| Age group | 50-60 | 40-50 | 40-50 |
| Gender | Male | Male | Male |
| Medical condition | NA | Pain | Anxiety |
| treated by cannabis | | | |

The present invention demonstrates the effect of consumption of different strains of cannabis on subjects, comparing relative levels of observed compounds, all measured on the same instrument. Comparison of compounds at different time points post-consumption allow us to understand the compounds' kinetics, which can indicate timing of consumption.

TABLE 2
Consumption and sampling details

| | | Subject #1 | | |
|---|---|---|---|---|
| | Control | First | Second | Subject #2 |
| | Subject | consumption | consumption | Consumption |
| THC (%) in the | 0 | 27.00 | 23.52 | 9.80 |
| product | | | | |
| CBD (%) in the | 0 | 0.10 | 0.06 | 7.30 |
| product | | | | |
| Consumption time* | NA | −4 hr | 0 min | 0 min |
| Consumption | NA | Vape | Vape | Vape |
| method | | | | |

*Time 0 was set to consumption prior to first post sampling (10 min) as described in FIG 14.

The preceding example(s) can be repeated with similar success by substituting the various components and configurations for others discussed elsewhere in this application.

### Embodiments

Embodiment 1: A method for detecting biomarkers in response to intoxicant consumption, the method comprising: receiving, by the one or more computing devices, a genetic and chemical profile of a cultivar; receiving, by the one or more computing devices, a user profile, wherein the user profile comprises: method of consumption and demographic data of a consumer; receiving, by the one or more computing devices, a metabolomic profile of said consumer, wherein the metabolomic profile comprises metabolomics data acquired at a time prior to consumption of said cultivar, at a time of consumption of said cultivar, and at a time after the consumption of said cultivar; comparing, by the one or more computing devices, said metabolomics data acquired at a time prior to consumption of said cultivar to said metabolomics data acquired at a time of consumption of said cultivar, and said metabolomics data acquired at a time after the consumption of said cultivar; generating, by the one or more computing devices, from said comparing step, a differences profile comprising differences recognized by the one or more computing devices in said metabolomics data acquired at said time prior to consumption of said cultivar to said metabolomics data acquired at said time of consumption of said cultivar, and said metabolomics data acquired at said time after the consumption of said cultivar; and calculating, by the one or more computing devices, a correlation between said time of consumption of said cultivar and one or more data points from said genetic and chemical profile of said cultivar, said user profile, or said metabolomics data of said differences profile.

Embodiment 2: The method of embodiment 1, wherein said metabolomic profile includes a plurality of ratios of biomarkers within the metabolomic profile.

Embodiment 3: The method of embodiment 1, wherein the calculating step comprises: training, a machine learning algorithm to estimate said time of consumption of said cultivar using said genetic and chemical profile of said cultivar, said user profile, and said metabolomics data of said differences profile; using said machine learning algorithm to estimate an unknown time of consumption of an unknown cultivar using new data comprising a plurality of data points from said user profile, and said metabolomics data of said differences profile; and generating a confidence score confidence score that represents the likelihood that a consumer consumed marijuana or another intoxicant within a specific time frame.

Embodiment 4: The method of embodiment 3, further comprising a step of feature extraction.

Embodiment 5: A system comprising: one or more computers and one or more storage devices storing instructions that are operable, when executed by the one or more computers, to cause the one or more computers to perform operations comprising: receiving, by the one or more computing devices, a genetic and chemical profile of a cultivar; receiving, by the one or more computing devices, a user profile, wherein the user profile comprises: method of consumption and demographic data of a consumer; receiving, by the one or more computing devices, a metabolomic profile of said consumer, wherein the metabolomic profile comprises metabolomics data acquired at a time prior to consumption of said cultivar, at a time of consumption of said cultivar, and at a time after the consumption of said cultivar; comparing, by the one or more computing devices, said metabolomics data acquired at a time prior to consumption of said cultivar to said metabolomics data acquired at a time of consumption of said cultivar, and said metabolomics data acquired at a time after the consumption of said cultivar; generating, by the one or more computing devices, from said comparing step, a differences profile comprising differences recognized by the one or more computing devices in said metabolomics data acquired at said time prior to consumption of said cultivar to said metabolomics data acquired at said time of consumption of said cultivar, and said metabolomics data acquired at said time after the consumption of said cultivar; and calculating, by the one or more computing devices, a correlation between said time of consumption of said cultivar and one or more data points from said genetic and chemical profile of said cultivar, said user profile, or said metabolomics data of said differences profile.

Embodiment 6: The system of embodiment 5, wherein said metabolomic profile includes a plurality of ratios of biomarkers within the metabolomic profile.

Embodiment 7: The system of embodiment 5, wherein the calculating step comprises: training, a machine learning algorithm to estimate said time of consumption of said cultivar using said genetic and chemical profile of said cultivar, said user profile, and said metabolomics data of said differences profile; using said machine learning algorithm to estimate an unknown time of consumption of an unknown cultivar using new data comprising a plurality of data points from said user profile, and said metabolomics data of said differences profile; and generating a confidence score confidence score that represents the likelihood that a consumer consumed marijuana or another intoxicant within a specific time frame.

Embodiment 8: The system of embodiment 7, further comprising a step of feature extraction.

Embodiment 9: A non-transitory computer-readable medium storing software comprising instructions executable by one or more computers which, upon such execution, cause the one or more computers to perform operations comprising: receiving, by the one or more computing devices, a genetic and chemical profile of a cultivar; receiving, by the one or more computing devices, a user profile, wherein the user profile comprises: method of consumption and demographic data of a consumer; receiving, by the one or more computing devices, a metabolomic profile of said consumer, wherein the metabolomic profile comprises metabolomics data acquired at a time prior to consumption of said cultivar, at a time of consumption of said cultivar, and at a time after the consumption of said cultivar; comparing, by the one or more computing devices, said metabolomics data acquired at a time prior to consumption of said cultivar to said metabolomics data acquired at a time of consumption of said cultivar, and said metabolomics data acquired at a time after the consumption of said cultivar; generating, by the one or more computing devices, from said comparing step, a differences profile comprising differences recognized by the one or more computing devices in said metabolomics data acquired at said time prior to consumption of said cultivar to said metabolomics data acquired at said time of consumption of said cultivar, and said metabolomics data acquired at said time after the consumption of said cultivar; and calculating, by the one or more computing devices, a correlation between said time of consumption of said cultivar and one or more data points from said genetic and chemical profile of said cultivar, said user profile, or said metabolomics data of said differences profile.

Embodiment 10: The non-transitory computer-readable medium of embodiment 9, wherein said metabolomic profile includes a plurality of ratios of biomarkers within the metabolomic profile.

Embodiment 11: The non-transitory computer-readable storage medium of embodiment 9, wherein the calculating step comprises: training, a machine learning algorithm to estimate said time of consumption of said cultivar using said genetic and chemical profile of said cultivar, said user profile, and said metabolomics data of said differences profile; using said machine learning algorithm to estimate an unknown time of consumption of an unknown cultivar using new data comprising a plurality of data points from said user profile, and said metabolomics data of said differences profile; and generating a confidence score confidence score that represents the likelihood that a consumer consumed marijuana or another intoxicant within a specific time frame.

Embodiment 12: The non-transitory computer-readable storage medium of embodiment 11, further comprising a step of feature extraction.

Embodiment 13: A method for detecting biomarkers in response to intoxicant consumption, the method comprising: receiving, by the one or more computing devices, a user profile, wherein the user profile comprises: method of consumption and demographic data of a consumer; receiving, by the one or more computing devices, a metabolomic profile of a consumer, wherein the metabolomic profile comprises metabolomics data acquired at a time prior to consumption of a substance, and at a time after the consumption of the substance; comparing, by the one or more computing devices, the metabolomics data acquired at the time prior to consumption of the substance to the metabolomics data acquired at the time after the consumption of the substance; generating, by the one or more computing devices, from the comparing step, a differences profile comprising differences recognized by the one or more computing devices in the metabolomics data acquired at the time prior to consumption of the substance to the metabolomics data acquired at the time after the consumption of said cultivar; and calculating, by the one or more computing devices, a correlation between the time of consumption of the substance and the differences profile.

Embodiment 14: The method of embodiment 13, further comprising: receiving, by the one or more computing devices a chemical profile of a substance.

Embodiment 15: The calculating step of embodiment 14, wherein the correlation is between the time of consumption of the substance, the differences profile, the chemical profile of the substance, and combinations thereof.

Embodiment 16: The method of embodiment 13, further comprising: receiving, by the one or more computing devices a genetic profile of a substance.

Embodiment 17: The calculating step of embodiment 16, wherein the correlation is between the time of consumption of the substance, the differences profile, the genetic profile of the substance, and combinations thereof.

Embodiment 18: The method of embodiment 13, wherein the metabolomic profile includes a plurality of ratios of biomarkers within the metabolomic profile.

Embodiment 19: The method of embodiment 13, wherein the calculating step comprises: training, a machine learning algorithm to estimate said time of consumption of the substance using the user profile, and the metabolomics data of the differences profile.

Embodiment 20: The method of embodiment 19, wherein the training uses a chemical profile of the substance.

Embodiment 21: The method of embodiment 20, wherein the training uses a genetic profile of the substance.

Embodiment 22: The method of embodiment 19, further comprising: Receiving a saliva sample from a target user; and Predicting, from the machine learning algorithm, a predicted time of consumption of an unknown substance using the saliva sample.

Embodiment 23: The method of embodiment 22, further comprising: comparing the saliva sample from the target user to a preselected threshold value and generating a impact value.

Embodiment 24: A system comprising: one or more computers and one or more storage devices storing instructions that are operable, when executed by the one or more computers, to cause the one or more computers to perform operations comprising: receiving, by the one or more computing devices, a user profile, wherein the user profile comprises: method of consumption and demographic data of a consumer; receiving, by the one or more computing devices, a metabolomic profile of a consumer, wherein the metabolomic profile comprises metabolomics data acquired at a time prior to consumption of a substance, and at a time after the consumption of the substance; comparing, by the one or more computing devices, the metabolomics data acquired at the time prior to consumption of the substance to the metabolomics data acquired at the time after the consumption of the substance; generating, by the one or more computing devices, from the comparing step, a differences profile comprising differences recognized by the one or more computing devices in the metabolomics data acquired at the time prior to consumption of the substance to the metabolomics data acquired at the time after the consumption of said cultivar; and calculating, by the one or more computing devices, a correlation between the time of consumption of the substance and the differences profile.

Embodiment 25: The system of embodiment 24, further comprising: receiving, by the one or more computing devices a chemical profile of a substance.

Embodiment 26: The system of embodiment 25, wherein the correlation is between the time of consumption of the substance, the differences profile, the chemical profile of the substance, and combinations thereof.

Embodiment 27: The system of embodiment 24, wherein the metabolomic profile includes a plurality of ratios of biomarkers within the metabolomic profile.

Embodiment 28: The system of embodiment 24, wherein the calculating step comprises: training, a machine learning algorithm to estimate said time of consumption of the substance using the user profile, and the metabolomics data of the differences profile.

Embodiment 29: The system of embodiment 28, wherein the training uses a chemical profile of the substance.

Embodiment 30: The system of embodiment 29, wherein the training uses a genetic profile of the substance.

Embodiment 31: The system of embodiment 28, further comprising: receiving a saliva sample from a target user; and predicting, from the machine learning algorithm, a predicted time of consumption of an unknown substance using the saliva sample.

Embodiment 32: The system of embodiment 31, further comprising: comparing the saliva sample from the target user to a preselected threshold value and generating a impact value.

Embodiment 33: A non-transitory computer-readable medium storing software comprising instructions executable by one or more computers which, upon such execution, cause the one or more computers to perform operations comprising: receiving, by the one or more computing devices, a user profile, wherein the user profile comprises: method of consumption and demographic data of a consumer; receiving, by the one or more computing devices, a metabolomic profile of a consumer, wherein the metabolomic profile comprises metabolomics data acquired at a time prior to consumption of a substance, and at a time after the consumption of the substance; comparing, by the one or more computing devices, the metabolomics data acquired at the time prior to consumption of the substance to the metabolomics data acquired at the time after the consumption of the substance; generating, by the one or more computing devices, from the comparing step, a differences profile comprising differences recognized by the one or more computing devices in the metabolomics data acquired at the time prior to consumption of the substance to the metabolomics data acquired at the time after the consumption of said cultivar; and calculating, by the one or more computing devices, a correlation between the time of consumption of the substance and the differences profile.

Embodiment 34: The non-transitory computer-readable medium of embodiment 33, further comprising: receiving, by the one or more computing devices a chemical profile of a substance.

Embodiment 35: The non-transitory computer-readable medium of embodiment 34, wherein the correlation is between the time of consumption of the substance, the differences profile, the chemical profile of the substance, and combinations thereof.

Embodiment 36: The non-transitory computer-readable medium of embodiment 33, wherein the metabolomic profile includes a plurality of ratios of biomarkers within the metabolomic profile.

Embodiment 37: The non-transitory computer-readable medium of embodiment 33, wherein the calculating step comprises: training, a machine learning algorithm to estimate said time of consumption of the substance using the user profile, and the metabolomics data of the differences profile.

Embodiment 38: The non-transitory computer-readable medium of embodiment 37, wherein the training uses a chemical profile of the substance.

Embodiment 39: The non-transitory computer-readable medium of embodiment 38, wherein the training uses a genetic profile of the substance.

Embodiment 40: The non-transitory computer-readable medium of embodiment 37, further comprising: receiving a saliva sample from a target user; and predicting, from the machine learning algorithm, a predicted time of consumption of an unknown substance using the saliva sample.

Embodiment 41: The non-transitory computer-readable medium of embodiment 40, further comprising: comparing the saliva sample from the target user to a preselected threshold value and generating a impact value.

Embodiment 42: A method comprising: receiving user information about a target user, the user information describing a metabolic profile associated with the target user; retrieving metabolomic information describing a plurality of metabolomic groups; retrieving global information describing a plurality of users in a database, each user associated with one of the metabolomic groups; identifying, for each user of the plurality of users, features describing at least demographic data of the user, and a corresponding metabolomic group of the user based on the global information; training one or more models using the identified features, the one or more models used by a plurality of classifiers, each classifier associated with a metabolomic group of the plurality of metabolomic groups and configured to determine a probability that the target user belongs to the metabolomic group; and predicting, from the plurality of metabolomic groups, a predicted metabolomic group of the target user using the plurality of classifiers and the user information.

Embodiment 43: The method of claim 1, wherein the features further describe a time of consumption of a substance.

Embodiment 44: The method of claim 1, wherein the features further describe a chemical profile of a cultivar consumed by the user.

Embodiment 45: The method of claim 44, wherein the features further describe a metabolomics information of a user acquired at a time prior to consumption of the cultivar, at a time of consumption of the cultivar, and at a time after the consumption of the cultivar.

Although the invention has been described in detail with particular reference to some embodiments, other embodiments can achieve the same or similar results. Upon studying this application, those skilled in the art will realize other equivalent variations and/or modifications that can also be used. It is intended that the claims contained in any patent issued on this application cover all such equivalents. The entire disclosures of all references, applications, patents, and publications cited above are hereby incorporated herein by reference.

### REFERENCES

Ishikawa, Shigeo, et al. “Identification of salivary metabolomic biomarkers for oral cancer screening.” Scientific reports 6 (2016).

Nielsen, Kirstine L., et al. “A metabolomics study of retrospective forensic data from whole blood samples of humans exposed to 3,4-methylenedioxymethamphetamine: a new approach for identifying drug metabolites and changes in metabolism related to drug consumption.” Journal of proteome research 15.2 (2016): 619-627.

Calderón-Santiago, M., et al. “Optimization study for metabolomics analysis of human sweat by liquid chromatography-tandem mass spectrometry in high resolution mode.” Journal of Chromatography A 1333 (2014): 70-78.

Sulek, Karolina, et al. “Hair metabolomics: identification of fetal compromise provides proof of concept for biomarker discovery.” Theranostics 4.9 (2014): 953.

Reo, Nicholas V. “NMR-based metabolomics.” Drug and chemical toxicology 25.4 (2002): 375-382.

Holmes, Elaine, et al. “The application of NMR-based metabonomics in neurological disorders.” NeuroRx 3.3 (2006): 358-372.

Plumb, Robert S., et al. “A rapid screening approach to metabonomics using UPLC and oa-TOF mass spectrometry: application to age, gender and diurnal variation in normal/Zucker obese rats and black, white and nude mice.” Analyst 130.6 (2005): 844-849.

Lindon, John C., et al. “Summary recommendations for standardization and reporting of metabolic analyses.” Nature biotechnology 23.7 (2005): 833-839.

Fiehn, Oliver. “Metabolomics—the link between genotypes and phenotypes.” Plant molecular biology 48.1-2 (2002): 155-171.

Deming, Stanley N. “Chemometrics: an overview.” Clinical chemistry 32.9 (1986): 1702-1706.

Lavine, Barry, and Jerome Workman. “Chemometrics.” Analytical chemistry 80.12 (2008): 4519-4531.

Crockford, D. J., et al. “Curve-fitting method for direct quantitation of compounds in complex biological mixtures using 1H NMR: application in metabonomic toxicology studies.” Analytical Chemistry 77.14 (2005): 4556-4562.

Dieterle, Frank, et al. “Probabilistic quotient normalization as robust method to account for dilution of complex biological mixtures. Application in 1H NMR metabonomics.” Analytical chemistry 78.13 (2006): 4281-4290.

### APA

Warrack, Bethanne M., et al. “Normalization strategies for metabonomic analysis of urine samples.” Journal of Chromatography B 877.5 (2009): 547-552.

Sysi-Aho, Marko, et al. “Normalization method for metabolomics data using optimal selection of multiple internal standards.” BMC bioinformatics 8.1 (2007): 93.

Want, Elizabeth J., et al. “Phospholipid capture combined with non-linear chromatographic correction for improved serum metabolite profiling.” Metabolomics 2.3 (2006): 145-154.

Sturm, Marc, et al. “OpenMS-an open-source software framework for mass spectrometry.” BMC bioinformatics 9.1 (2008): 163.

Yu, Tianwei, et al. “apLCMS-adaptive processing of high-resolution LC/MS data.” Bioinformatics 25.15 (2009): 1930-1936.

Wishart, David S., et al. “HMDB: the human metabolome database.” Nucleic acids research 35.suppl 1 (2007): D521-D526.

Smith, Colin A., et al. “METLIN: a metabolite mass spectral database.” Therapeutic drug monitoring 27.6 (2005): 747-751.

Austin, Christopher P., et al. “NIH molecular libraries initiative.” Science 306.5699 (2004): 1138-1139.

Dufresnes, Christophe, et al. “Broad-Scale Genetic Diversity of Cannabis for Forensic Applications.” PloS one 12.1 (2017): e0170522.

Coyle, H. Miller, et al. “An overview of DNA methods for the identification and individualization of marijuana.” Croatian medical journal 44.3 (2003): 315-321.

Lata, Hemant, et al. “Molecular analysis of genetic fidelity in Cannabis sativa L. plants grown from synthetic (encapsulated) seeds following in vitro storage.” Biotechnology letters 33.12 (2011): 2503-2508.

Bailey, Keith. “The value of the Duquenois test for cannabis—a survey.” Journal of Forensic Science 24.4 (1979): 817-841.

Hazekamp, Arno, et al. “Cannabis: From Cultivar to Chemovar II-A Metabolomics Approach to Cannabis Classification.” Cannabis and Cannabinoid Research 1.1 (2016): 202-215.

Dufresnes, Christophe, et al. “Broad-Scale Genetic Diversity of Cannabis for Forensic Applications.” PloS one 12.1 (2017): e0170522.

Chapman, C. Richard, Karen Syrjala, and Mukund Sargur. “Pain as a manifestation of cancer treatment.” Seminars in oncology nursing. Vol. 1. No. 2. WB Saunders, 1985.

Melzack, Ronald. “The McGill Pain Questionnaire: major properties and scoring methods.” Pain 1.3 (1975): 277-299.

Rohlf, F. James. NTSYS-pc: numerical taxonomy and multivariate analysis system. Applied Biostatistics, 1992.

Dunn, G., and B. S. Everitt. “An Introduction to Mathematical Taxonomy Cambridge University Press Cambridge.” UK Google Scholar (1982).

Excoffier, Laurent, Peter E. Smouse, and Joseph M. Quattro. “Analysis of molecular variance inferred from metric distances among DNA haplotypes: application to human mitochondrial DNA restriction data.” Genetics 131.2 (1992): 479-491.

Peakall, R. O. D., and Peter E. Smouse. “GENALEX 6: genetic analysis in Excel. Population genetic software for teaching and research.” Molecular ecology notes 6.1 (2006): 288-295.

Soga, Tomoyoshi, et al. “Quantitative metabolome analysis using capillary electrophoresis mass spectrometry.” Journal of proteome research 2.5 (2003): 488-494.

Patkar, Ashwin A., et al. “Alterations in tryptophan and purine metabolism in cocaine addiction: a metabolomic study.” Psychopharmacology 206.3 (2009): 479-489.

Prescot, Andrew P., et al. “Neurochemical alterations in adolescent chronic marijuana smokers: a proton MRS study.” Neuroimage 57.1 (2011): 69-75.

What is claimed is:

1. A method for detecting biomarkers in response to intoxicant consumption, the method comprising:
receiving, by the one or more computing devices, a user profile, wherein the user profile comprises: method of consumption and demographic data of a consumer;
receiving, by the one or more computing devices, a metabolomic profile of a consumer, wherein the metabolomic profile comprises metabolomics data acquired at a time prior to consumption of a substance, and at a time after the consumption of the substance;
comparing, by the one or more computing devices, the metabolomics data acquired at the time prior to consumption of the substance to the metabolomics data acquired at the time after the consumption of the substance;
generating, by the one or more computing devices, from the comparing step, a differences profile comprising differences recognized by the one or more computing devices in the metabolomics data acquired at the time prior to consumption of the substance to the metabolomics data acquired at the time after the consumption of the substance; and
calculating, by the one or more computing devices, a correlation between the time of consumption of the substance and the differences profile.

2. The method of claim 1, further comprising: receiving, by the one or more computing devices a chemical profile of a substance.

3. The method of claim 2, wherein the correlation is between the time of consumption of the substance, the differences profile, the chemical profile of the substance, and combinations thereof.

4. The method of claim 1, further comprising: receiving, by the one or more computing devices a genetic profile of a substance.

5. The method of claim 4, wherein the correlation is between the time of consumption of the substance, the differences profile, the genetic profile of the substance, and combinations thereof.

6. The method of claim 1, wherein the metabolomic profile includes a plurality of ratios of biomarkers within the metabolomic profile.

7. The method of claim 1, wherein the calculating step comprises:
training, a machine learning algorithm to estimate the time of consumption of the substance using the user profile, and the metabolomics data of the differences profile.

8. The method of claim 7, wherein the training uses a chemical profile of the substance.

9. The method of claim 8, wherein the training uses a genetic profile of the substance.

10. The method of claim 7, further comprising:
receiving a saliva sample from a target user; and
predicting, from the machine learning algorithm, a predicted time of consumption of an unknown substance using the saliva sample.

11. The method of claim 10, further comprising: comparing the saliva sample from the target user to a preselected threshold value and generating an impact value.

12. A system comprising:
one or more computers and one or more storage devices storing instructions that are operable, when executed by the one or more computers, to cause the one or more computers to perform operations comprising:
receiving, by the one or more computing devices, a user profile, wherein the user profile comprises: method of consumption and demographic data of a consumer;
receiving, by the one or more computing devices, a metabolomic profile of a consumer, wherein the metabolomic profile comprises metabolomics data acquired at a time prior to consumption of a substance, and at a time after the consumption of the substance;
comparing, by the one or more computing devices, the metabolomics data acquired at the time prior to consumption of the substance to the metabolomics data acquired at the time after the consumption of the substance;
generating, by the one or more computing devices, from the comparing step, a differences profile comprising differences recognized by the one or more computing devices in the metabolomics data acquired at the time prior to consumption of the substance to the metabolomics data acquired at the time after the consumption of the substance; and
calculating, by the one or more computing devices, a correlation between the time of consumption of the substance and the differences profile.

13. The system of claim 12, further comprising: receiving, by the one or more computing devices a chemical profile of a substance.

14. The system of claim 13, wherein the correlation is between the time of consumption of the substance, the differences profile, the chemical profile of the substance, and combinations thereof.

15. The system of claim 12, wherein the metabolomic profile includes a plurality of ratios of biomarkers within the metabolomic profile.

16. The system of claim 12, wherein the calculating step comprises:
training, a machine learning algorithm to estimate the time of consumption of the substance using the user profile, and the metabolomics data of the differences profile.

17. The system of claim 16, wherein the training uses a chemical profile of the substance.

18. The system of claim 17, wherein the training uses a genetic profile of the substance.

19. The system of claim 16, further comprising:
receiving a saliva sample from a target user; and
predicting, from the machine learning algorithm, a predicted time of consumption of an unknown substance using the saliva sample.

20. The system of claim 19, further comprising: comparing the saliva sample from the target user to a preselected threshold value and generating an impact value.
